(19) United States
(12) Patent Application Publication
(10) Pub. No.: US 2005/0042614 A1
(43) Pub. Date: Feb. 24, 2005

(54) Methods to treat diabetes and related conditions based on polymorphisms in the tcf-1 gene

(76) Inventors: Thomas Edward Hughes, Concord, MA (US); Christian Nicolas Lavedan, Potomac, MD (US); Milhael Hristos Polymeropoulos, Potomac, MD (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/493,885

(22) PCT Filed: Oct. 30, 2002
(86) PCT No.: PCT/EP02/12113

Related U.S. Application Data
(60) Provisional application No. 60/335,513, filed on Oct. 31, 2001.

(51) Int. Cl. C12Q 1/68

(52) U.S. Cl. 435/6

(57) ABSTRACT

This invention relates to the use of the novel association between the 483 A>G single nucleotide polymorphism of the TCF1 gene and the clinical response to glycemic control agents, such as DPPIV inhibitors, in patients with disorders of glycemic control, especially diabetes and impaired glucose metabolism. This invention provides methods to classify patients for treatment and/or for optimization of clinical studies and to treat patients based on this association.

### BACKGROUND OF THE INVENTION

1. Field of the Invention

This invention relates to methods to treat disorders characterized by impaired glycemic control, especially Diabetes Mellitus and related conditions. In particular, this invention relates to the use of genomic analysis to determine a subject's responsiveness to glycemic control agents such as dipeptidylpeptidase IV (DPP4) inhibitors and other glycemic control methods and strategies, including the timing of initiation of treatment and the selection of optimum agents. treatment regimens, and dosages.

2. Description of the Related Art

Diabetes Mellitus is one form of a broad group of disorders in humans, characterized by impaired glycemic control or impaired control of blood glucose levels. Diabetes itself is a chronic hormonal disorder characterized by impaired metabolism of glucose and other energy yielding fuels, as well as the late development of serious vascular and neuropathic complications. Diabetes accounts for nearly 15% of healthcare costs in the U.S. and is the leading cause of blindness among working-age people as well as end-stage renal disease (ESRD) and non-traumatic limb amputations. Diabetes increases the risk of cardiac, cerebral and peripheral vascular disease 2- to 7-fold and it is a major cause of neonatal morbidity and mortality.

Diabetes is a complex and diverse group of disorders but all forms are associated with a common hormonal defect, i.e., insulin deficiency. This deficiency may be total, partial or relative when viewed in the context of co-existing insulin resistance. Relative or absolute insulin deficiency plays a primary role in the metabolic derangement linked to diabetes and the resulting hyperglycemia in turn plays a key role in the numerous complications of the disease.

Classification

The newly revised classification of diabetes mellitus is summarized in Table 1. Clinical diabetes may be divided into four general subclasses, including (1) type 1 (caused by beta cell destruction and characterized by absolute insulin deficiency), (2) type 2 (characterized by insulin resistance and relative insulin deficiency), (3) other specific types of diabetes (associated with various identifiable clinical conditions or syndromes), and (4) gestational diabetes mellitus. In addition to these clinical categories, two conditions—impaired glucose tolerance and impaired fasting glucose—refer to a metabolic state intermediate between normal glucose homeostasis and overt diabetes. These conditions significantly increase the later risk of diabetes mellitus and may in some instances be part of its natural history. It should be noted that patients with any form of diabetes might require insulin treatment at some point. For this reason the previously used terms insulin-dependent diabetes (for type 1 diabetes mellitus) and non-insulin-dependent diabetes (for type 2) have been eliminated.

TABLE 1
Classification of diabetes

Clinical diabetes

| I. | Type 1 diabetes, formerly called insulin-dependent diabetes mellitus | |
|---|---|---|
| | (IDDM) or “juvenile-onset diabetes | |
| | A. | Immune mediated |
| | B. | Idiopathic |
| II. | Type 2 diabetes, formerly called non-insulin-dependent diabetes | |
| | (NIDDM) or “adult-onset diabetes” | |
| III. | Other specific types | |
| | A. | Genetic defects of β-cell function (e.g., maturity-onset diabetes |
| | | of the young [MODY] types 1-3 and point mutations in mito- |
| | | chondrial DNA) |
| | B. | Genetic defects in insulin action |
| | C. | Disease of the exocrine pancreas (e.g., pancreatitis, trauma, |
| | | pancreatectomy, neoplasia, cystic fibrosis, hemocrhomatosis, |
| | | fibrocalculous pancreatopathy) |
| | D. | Endocrinopathies (e.g., acromegaly, Cushing's syndrome, hyper- |
| | | thyroidism, pheochromocytoma, glucagonoma, somatostinoma, |
| | | aldosteronoma) |
| | E. | Drug or chemical induced (e.g., glucocorticosteroids, thiazides, |
| | | diazoxide, pentamidine, vacor, thyroid hormone, phenytoin |
| | | [Dilantin], β-agonists, oral contraceptives) |
| | F. | Infections (e.g., congenital rubella, cytomegalovirus) |
| | G. | Uncommon forms of immune-mediated diabetes (e.g., “stiff- |
| | | man” syndrome, anti-insulin receptor antibodies) |
| | H. | Other genetic syndromes (e.g., Down, Klinefelter's, Turner's |
| | | syndrome, Huntington's disease, myotonic dystrophy, lipo- |
| | | dystrophy, ataxia-telangiectasia) |

| | |
|---|---|
| IV. | Gestational diabetes mellitus |

Risk categories

| | |
|---|---|
| I. | Impaired fasting glucose |
| II. | Impaired glucose tolerance |

Type 1 Diabetes Mellitus

Patients with this disorder have little or no insulin secretory capacity and depend on exogenous insulin to prevent metabolic decompensation (e.g., ketoacidosis) and death. Commonly but not always, diabetes appears abruptly (i.e., over days and weeks) in previously healthy non-obese children or young adults; in older age groups it may have a more gradual onset. At the time of initial evaluation the typical patient often appears ill, has marked symptoms (e.g., polyuria, polydipsia, polyphagia, and weight loss), and may demonstrate ketoacidosis. Type 1 diabetes is believed to have a long asymptomatic pre-clinical stage often lasting years, during which pancreatic beta cells are gradually destroyed by an autoimmune attack that is influenced by HLA and other genetic factors, as well as the environment. Initially, insulin therapy is essential to restore metabolism toward normal. However, a so-called honeymoon period may follow and last weeks or months, during which time smaller doses of insulin are required because of partial recovery of beta cell function and reversal of insulin resistance caused by acute illness. Thereafter, insulin secretory capacity is gradually lost (over several years). The association of type 1 diabetes with specific immune response (HLA) genes and the presence of antibodies to islet cells and their constituents provides strong support for the theory that type 1 diabetes is an autoimmune disease. This syndrome accounts for less than 10% of diabetes in the United States.

Type 2 Diabetes Mellitus

Type 2, by far the most common form of the disease, is found in over 90% of the diabetic patient population. These patients retain a significant level of endogenous insulin secretory capacity. However, insulin levels are low relative to the magnitude of insulin resistance and ambient glucose levels. Type 2 patients are not dependent on insulin for immediate survival and ketosis rarely develops, except under conditions of great physical stress. Nevertheless, these patients may require insulin therapy to control hyperglycemia. Type 2 diabetes typically appears after the age of 40 years, has a high rate of genetic penetrance unrelated to HLA genes, and is associated with obesity. The clinical features of type 2 diabetes may be mild (fatigue, weakness, dizziness, blurred vision, or other non-specific complaints may dominate the picture) or may be tolerated for many years before the patient seeks medical attention. Moreover, if the level of hyperglycemia is insufficient to produce symptoms, the disease may become evident only after complications develop.

Other Specific Type of Diabetes

This category encompasses a variety of diabetic syndromes attributed to a specific disease, drug, or condition. Genetic research has provided new insights into the pathogenesis of MODY, which was formerly included as a form of type 2 diabetes. MODY encompasses several genetic defects of beta cell function, among which mutations at several genetic loci on different chromosomes have been identified. The most common forms—MODY type 3—is associated with a mutation for a transcription factor encoded on chromosome 12 named hepatocyte nuclear factor 1α (HNF1, also known as TCF1) and—MODY type 2 is associated with mutations of the glucokinase gene (on chromosome 7). Mutations of the HNF-4α gene (on chromosome 20) are responsible for type 1 of MODY. Each of these conditions is inherited in an autosomal dominant pattern. Two new rare forms of MODY are associated with mutations of the HNF-1β (on chromosome 17) and an insulin gene transcription factor termed PDX-1 or 1DX-1 (on chromosome 13).

The distinction between the various subclasses of diabetes mellitus is usually made on clinical grounds. However, a small subgroup of patients are difficult to classify, that is, they display features common to both type 1 and 2 diabetes. Such patients are commonly non-obese and have reduced insulin secretory capacity that is not sufficient to make them ketosis prone. Many initially respond to oral agents but, with time, require insulin. Some appear to have a slowly evolving form of type 1 diabetes, whereas others defy easy categorization.

Gestational Diabetes

The term gestational diabetes describes women with impaired glucose tolerance that appears or is first detected during pregnancy. Gestational diabetes usually appears in the 2^{nd} or 3^{rd} trimester, a time when pregnancy-associated insulin antagonistic hormones peak. After delivery, glucose tolerance generally (but not always) reverts to normal.

Diagnosis

The diagnosis of diabetes is usually straightforward when the classic symptoms of polyuria, polydipsia, and weight loss are present. All that is required is a random plasma glucose measurement from venous blood that is 200 mg/dL or greater. If diabetes is suspected but not confirmed by a random glucose determination, the screening test of choice is overnight fasting plasma glucose level. The diagnosis is established if fasting glucose is equal to or greater than 126 mg/dL on at least two separate occasions.

Related Conditions

Impaired Glucose Tolerance and Impaired Fasting Glucose

Impaired glucose tolerance (IGT) and impaired fasting glucose (IFG) are terms applied to individuals who have glucose levels that are higher than normal, (under fed or fasting conditions, respectively) but lower than those accepted as diagnostic for diabetes mellitus. Both conditions are associated with an increased risk for cardiovascular disease, but do not produce the classic symptoms or the microvascular and neuropathic complications associated with diabetes mellitus. In a subgroup of patents (about 25 to 30%), however, type 2 diabetes eventually develops.

Impaired Glucose Metabolism

Impaired Glucose Metabolism (IGM) is defined by blood glucose levels that are above the normal range but are not high enough to meet the diagnostic criteria for type 2 diabetes mellitus. The incidence of IGM varies from country to country, but usually occurs 2-3 times more frequently than overt diabetes. Until recently, individuals with IGM were felt to be pre-diabetics, but data from several epidemiological studies argue that subjects with IGM are heterogeneous with respect to their risk of diabetes and their risk of cardiovascular morbidity and mortality. The data suggest that subjects with IGM, in particular, those with impaired glucose tolerance (IGT), do not always develop diabetes, but whether they are diabetic or not, they are, nonetheless, at high risk for cardiovascular morbidity and mortality. Among subjects with IGM, about 58% have Impaired Glucose Tolerance (IGT), another 29% have impaired fasting glucose (IFG), and 13% have both abnormalities (IFG/IGT). As discussed above, IGT is characterized by elevated post-prandial (post-meal) hyperglycemia while IFG has been defined by the ADA on the basis of fasting glycemic values.

The categories of (a) normal glucose tolerance (NGT), (b) impaired glucose metabolism (IGM) and (c) overt type 2 diabetes mellitus were defined by the ADA in 1997 as follows:
(a) Normal Glucose Tolerance (NGT)=fasting plasma glucose level <6.1 mmol/L or less than 110 mg/dl and a 2 h post-prandial glucose level of <7.8 mmol/L or <140 mg/dl.
(b) Impaired Glucose Metabolism (IGM) is impaired fasting glucose (IFG) defined as IFG=fasting glucose level of 6.1-7 mmol/L or 140-220 mg/dl and/or impaired glucose tolerance (IGT)=a 2 h post-prandial glucose level (75 g OGTT) of 7.8-11.1 mmol/L or 140-220 mg/dl.
(c) Type 2 diabetes=fasting glucose of greater than 7 mmol/L or 126 mg/dl or a 2 h post-prandial glucose level (75 g OGTT) of greater than 11.1 mmol/L or 200 mg/dl.

These criteria were defined using the WHO recommended conditions for administration of an oral glucose tolerance test ((75 g OGTT), i.e., the oral administration of a glucose load containing the equivalent of 75 g of anhydrous glucose dissolved in water with a blood sample taken 2 hours later to analyze the post-prandial glucose. Other OGTT test conditions have confirmed the associated risks of the IGT and IFG categories including: 1) using 50 g glucose instead of 75 g, 2) using a casual (non-fasting) glucose sample as the analyte, and 3) analyzing the post-prandial glucose at 1 hour rather than 2 hours post-glucose load. Under all of these conditions, the glycemic categories defined above have been linked to the increased risks described below, but the standardized OGTT is preferred in order to minimize variations in test results.

Individuals with IGM, especially those with the subcategory IFG, are known to have a significantly higher rate of progression to diabetes than normoglycemic individuals and are known to be high at cardiovascular risk, especially if they develop diabetes. Interestingly, subjects with IGM, more specifically those with the subcategory IFG, have a high incidence of cancer, cardiovascular diseases and mortality even if they never develop diabetes. Therefore, IGM and more specifically, the subgroup IFG, appears to be at high cardiovascular risk, especially after patients become overtly diabetic. IGT also referred to as postprandial hyperglycemia (PPHG), on the other hand, is associated with a high risk for cancer, cardiovascular disease and mortality in non-diabetics and diabetics. See Hanefeld M and Temelkova-Kurktschiev T, Diabet. Med 1997; 14 Suppl. 3: S6-S11.

The increased risk associated with IGT is independent of all other known cardiovascular risk factors including age, sex, hypertension, low HDL and high LDL cholesterol levels See, Lancet 1999; 354: 617-621. In addition, epidemiological studies suggest that postprandial hyperglycaemia (PPHG) or hyperinsulinaemia are independent risk factors for the development of macro-vascular complications of diabetes mellitus. See, Mooradian A D and Thurman J E, Drugs 1999; 57(1):19-29. PPHG similiar to HbA1c has been corelated with the presence of diabetic complications, notably retinkopathy and nephropathy. See Pettitt D J et al. Lancet 1980; 2: 1050-2, Jarrett R J Lancet 1976; 2: 1009-2 and Teuscher A et al. Diabetes Care 1988; 11: 246-51.

One mechanism through which IGM, and more specifically, IGT, has been linked to micro- and macro-angiopathic complications in the absence of the abnormal FPG characteristic of diabetics, is postprandial hyperglycemia. Isolated postprandial hyperglycemia, even in non-diabetics, has been shown to reduce the natural free-radical trapping agents (TRAP) that are present in serum. Decreasing the level of TRAP has been shown, under experimental conditions, to be associated with an increase in free radical formation and increased oxidative stress. These free radicals have been implicated in the pathological microvascular and macro-vascular changes associated with atherosclerosis, cardiovascular morbidity and mortality, and cancer See, Ceriello, A, Diabetic Medicine 15: 188-193, 1998. The decrease of natural antioxidants like TRAP during post-prandial hyperglycemia may explain the increased cardiovascular risk in subjects with IGM, and specifically IGT, that do not develop diabetes.

The fact that IGT is an independent risk factor in non-diabetics as well as diabetics justifies it as a new indication, separate from diabetes, for prevention and treatment of cardiovascular morbidity and mortality as well as cancer. Thus, IGM is associated with following potential diseases or conditions: 1) progression to overt diabetes mellitus type 2 (Code 250.2 of the International Classification of Diseases 9th version=ICD-9 Code 250.2) [Diabetes Research and Clinical Practice 1998; 40: S 1-S2]; 2) increased microvascular complications of diabetes especially retinopathy and other ophthalmic complications of diabetes (ICD-9 code 250.5), nephropathy (ICD-9 code 250.4), neuropathy (ICD-9 code 250.6) [Diabetes Care 2000, 23: 1113-1118], and peripheral angiopathy or gangrene (ICD-9 code 250.7); 3) increased cardiovascular morbidity (ICD-9 codes 410-414) especially myocardial infarctions (ICD-9 code 410), coronary heart disease or atherosclerosis (ICD-9 code 414) and other acute and subacute forms of coronary ischemia (ICD-9 code 411); 4) excess cerebrovascular diseases like stroke (ICD-9 codes 430-438) [Circulation 1998, 98:2513-2519]); 5) increased cardiovascular mortality (ICD-9 codes 390-459) [Lancet 1999; 354: 617-621], and sudden death (ICD-9 code 798.1); 6) higher incidences and mortality rates of malignant neoplasms (ICD-9 codes 140-208) [Am J Epidemiol. 1990, 131: 254-262, Diabetologia 1999; 42: 1050-1054]. Other metabolic disturbances that are associated with IGIVI include dyslipidemia (ICD-9 code 272), hyperuricemia (ICD-9 code 790.6) as well as hypertension (ICD-9 codes 401-404) and angina pectoris (ICD-9 code 413.9) [Ann Int Med 1998, 128:524-533]. Clearly, the broad spectrum of diseases and conditions that are linked to IGM, and especially IGT, represents an area of tremendous medical need.

Many of the same diseases and conditions have been associated with both IGM and diabetes, but only recently has it been possible to identify that that the non-diabetic population that has IGM, and especially IGT, should be an indication for prevention and treatment. Accordingly, in subjects with IGM and especially IGT and/or IFG, the restoration of early phase insulin secretion and/or the reduction of prandial hyperglycemia should help to prevent or delay the progression to overt diabetes and to prevent or reduce microvascular complications associated with diabetes by preventing the development of the overt diabetes. In addition, in individuals with IGM and especially those with IGT and/or IFG, the restoration of early phase insulin secretion and/or reduction of post-prandial hyperglycemia should also prevent or reduce the excessive cardiovascular morbidity and mortality, and prevent cancer or reduce its mortality in individuals.

insulin Secretion and Action

Insulin is initially synthesized in the pancreatic beta cells as a large single-chain polypeptide, pro-insulin, and subsequent cleavage of pro-insulin results in the removal of a connecting strand (C peptide) and appearance of the smaller, double-chain insulin molecule (51 amino acid residues). The concentration of glucose is the key regulator of insulin secretion. For glucose to activate secretion, it must first be transported by a protein (GLUT 2) into the beta cell, phosphorylated by the enzyme glucokinase, and metabolized. The immediate triggering process is poorly understood but probably involves the activation of signal transduction pathways, closure of adenosine triphosphate (ATP)-sensitive potassium channels, and entry of calcium into the beta cell. Normally, when blood glucose rises even slightly above the fasting level of 75 to 100 mg/dL, beta cells secrete insulin, initially from pre-formed stored insulin and later from the synthesis of new insulin. The route of glucose entry as well as its concentration determines the magnitude of the response. Higher insulin levels are produced when glucose is given orally than when given intravenously because of the simultaneous release of gut peptides (e.g., glucagon-like peptide I, gastric inhibitory polypeptide). Other insulin secretagogues include amino acids and vagal stimulation. Once secreted into portal blood, insulin removes approximately 50% of the insulin and degrades it. The consequence of this uptake is that portal vein insulin is always at least two- to four-fold higher than that in the peripheral circulation. Conversely, when blood glucose levels decline even slightly (e.g., to 70 mg/dL), insulin secretion promptly diminishes.

Insulin acts on responsive tissues by first passing through the vascular compartment and, on reaching its target, binding to its specific receptor. The insulin receptor is a heterodimer with two α- and β-chains formed by disulfide bridges. The α-subunit resides on the extracellular surface and is the site of insulin binding. The β-subunit spans the membrane and can be phosphorylated on serine, threonine, and tyrosine residues on the cytoplasmic face. The intrinsic protein tyrosine kinase activity of the β-subunit is essential for insulin receptor function. Rapid receptor autophosphorylation and tyrosine phosphorylation of cellular substrates (e.g., insulin receptor substrates 1 and 2) are important early steps in insulin action. Thereafter, a series of phosphorylation and dephosphorylation reactions are triggered that ultimately produced insulin's effects in insulin-sensitive tissues (liver, muscle, and fat). A variety of post-receptor signal transduction pathways are activated by insulin, including Pl3 (phosphatidylinositol 3′) kinase, an enzyme that appears to be critical for the translocation of glucose transporters (GLUT 4) to the cell surface and, in turn, glucose uptake.

A number of other hormones termed counter-regulatory hormones (glucagon, growth hormone, catecholamines, and cortisol) oppose the metabolic actions of insulin. Among these, glucagon and to a lesser extent growth hormone have important roles in development of the diabetic syndrome. Glucagon is secreted by pancreatic alpha cells in response to hypoglycemia, amino acids, and activation of the autonomic nervous system. Its major effect is on the liver, where it stimulates glycogenolysis, gluconeogenesis, and ketogenesis via cyclic adenosine monophosphate-dependent mechanisms. It is normally inhibited by hyperglycemia but is absolutely or relatively increased in both type 1 and type 2 diabetes despite the presence of hyperglycemia.

Diabetes is characterized by marked post-prandial hyperglycemia after carbohydrate ingestion. In type 2 diabetes, the combined effects of delayed insulin secretion and hepatic insulin resistance impairs the suppression of hepatic glucose production and the ability of the liver to store glucose as glycogen. Hyperglycemia ensues, even though insulin levels may eventually rise to levels above those seen in non-diabetic individuals (insulin secretion remains deficient relative to the prevailing glucose level), because insulin resistance reduces the capacity of muscle to remove the excess glucose released from the liver and store it in the myocyte as glycogen.

The pharmacological treatment of diabetes mellitus has traditionally involved intervention with insulin or oral glucose-lowering drugs. In type 1 diabetes, the primary focus is to replace insulin secretion. In type 2 diabetes, the most well established treatment strategies aim to increase the secretion or physiological effects of insulin. This can be accomplished by stimulating insulin secretion directly with insulin secretogogues such as the sulfonylureas or benzoic acid derivatives, or by reducing peripheral insulin resistance with agents such as those represented by the PPARγ agonist thiazolidinedione class of drugs. In some type 2 diabetics, insulin itself is needed either early in the stabilization process or in combination with one or more of the other classes of drugs. For general review of diabetes see, Cecil Textbook of Medicine 21^{st} edition; Goldman, L. and Bennett J. C. Eds. Saunders Co. Phili (2000), esp. pages 1263-1285.

Several novel approaches to the treatment of diabetes employ the actions of Glucagon-Like-Peptide 1 (GLP-1). GLP-1 is a peptide hormone that is released into the bloodstream from the intestinal tract in response to a meal. GLP-1 has several actions that lower glucose levels, including acting directly on pancreatic beta cells to augment insulin release and promoting the synthesis of insulin. GLP-1 arises from tissue-specific post-translational processing of the glucagon precursor in the intestinal L-cell, see, Ørskov C. Diabetologia 35:701-711 (1992).

In healthy subjects, GLP-1 potently influences glycemic levels through a number of physiologic mechanisms including modulation of insulin and glucagon concentrations, see Ørskov C. Diabetologia 35:701-711 (1992); Holst J J, et al. In Glugagon III. Handbook of Experimental Pharmacology; Lefevbre P J. Ed. Berlin, Springer Verlag, 311-326 (1996); and Deacon C F, et al. Diabetes, Vol. 47:764-769 (1998). The pancreatic effects of GLP-1 are glucose dependent, see, Kregmann B, et al. Laneifii 1300-1304 (1987); Weir G C, Diabetes 38:338-342 (1989).

These same effects also occur in patients with diabetes and tend to normalize blood glucose levels in type 2 diabetes subjects and improve glycemic control in type 1 patients, see, Gutniak M, et al. N Engl J Med 236:1316-1322 (1992); Nathan D M, et al. Diabetes Care 15:270-276 (1992); and Nauck M A, et al. Diabetologia 36:741-744 (1993).

Both endogenous and exogenously administered GLP-1 are rapidly metabolized and have a plasma half-life (t_{1/2}) of only 1-2 minutes in vivo. The amino peptidase dipeptidylpeptidase IV (DPP4) is the primary cause of this rapid metabolism. DPP4 action on GLP-1 produces an NH₂-terminally truncated metabolite GLP-1 (9-36) amide, see, Kieffer T J, et al. Endocrinology 136:3585-3596 (1995); Mentlien R, et al. Eur J Biochem 214:829-635 (1993); Deacon C F, et al. J Clin Endocrinol Metab 80:952-957 (1995); Deacon C F, et al. Diabetes 44:1126-1131 (1995).

Dipeptidylpeptidase IV (DPP4; EC 3.4.14.5), is identical to ADA complexing protein-2 and to the T-cell activation antigen CD26. DPP4 is a serine exopeptidase that cleaves X-proline dipeptides from the N-terminus of polypeptides. It is an intrinsic membrane glycoprotein anchored into the cell membrane by its N-terminal end. High levels of the enzyme are found in the brush-border membranes of the kidney proximal tubule and of the small intestine, but several other tissues also express the enzyme. The enzyme is present in the fetal colon but disappears at birth. It is ectopically expressed in some human colon adenocarcinomas and human colon cancer cell lines. From such a colon cancer cell line, Darmoul, et al. Ann. Hum. Genet. 54: 191-197, (1990) isolated a cDNA probe for intestinal DPP4 and, by Southern analysis of somatic cell hybrids, assigned the gene to chromosome 2. This assignment was confirmed by Mathew, et al. Genomics 22: 211-212 (1994), who sublocalized the DPP4 gene to 2q23 by fluorescence in situ hybridization. Misumi, et al. Biochim. Biophys. Acta 1131: 333-336, (1992) isolated and sequenced the cDNA coding for DPP4. The nucleotide sequence (3,465 bp) of the cDNA contained an open reading frame encoding a polypeptide comprising 766 amino acids, 1 residue less than those of the rat protein. The predicted amino acid sequence exhibited 84.9% identity to that of the rat enzyme.

Abbott, et al. Immunogenetics 40: 331-338 (1994) demonstrated that CD26 spans approximately 70 kb and contains 26 exons, ranging in size from 45 bp to 1.4 kb. the nucleotides that encode the serine recognition site (G-W-S-Y-G) are split between 2 exons. This clearly distinguishes the genomic organization of the propyl oligopeptidase family from that of the classic serine protease family. CD26 encodes 2 messages sized at about 4.2 and 2.8 kb. These are both expressed at high levels in the placenta and kidney and at moderate levels in the lung and liver. Only the 4.2 kb mRNA was expressed at low levels in skeletal muscle, heart, brain, and pancreas. By fluorescence in situ hybridization, Abbott, et al. (1994), supra, mapped the gene to 2q24.3.

Any pharmaceutically viable DPP4 (DPP IV) inhibitor can be used to prolong the half-life and increase the action of GLP-1 in vivo. Several studies have found that the inhibition of DPP4 improves glucose homeostasis in rats and augments the in situ response to intravenous glucose load in pigs, see, Deacon F., et al. Diabetes 47:764-769 (1998); Pauly R P, et al. Regal Pept 643:148 (1996); Balkan B, et al. Diabetologia 40(Suppl 1)A131 (1997) and Li X, et al. Diabetes 46(Suppl 1):237A (1997).

In pig studies, the inhibition in vivo of DPP4 prevents the NH₂ terminal degradation of GLP-1, thus extending the t_{1/2} of the biologically active peptide. The presence of the DPP4 inhibitor potentiates both the in-situ response to intravenous glucose given with a GLP-1 infusion and also improves glucose tolerance seen after oral glucose without exogenous GLP-1 by enhancing the action of endogenous GLP-1, see, Deacon C F. Diabetes 47:764-769 (1998).

In other studies targeted inactivation of the DPP4 (or CD26) gene yielded healthy mice that had normal blood glucose levels in the fasted state but reduced glycemic excursion after a glucose challenge. See Marguet D, et al. Proc Natl Acad Sci USA 97:6874-6879 (2000). This group also found increased levels of glucose-stimulated circulating insulin and increased intact insulinotropic form of GLP-1 in mice with homozygous inactivated DPP4 gene.

The administration of a pharmacological inhibitor of DPP4 enzymatic activity was found to improve glucose tolerance in wild type but not in DPP4 gene inactivated mice. This DPP4 inhibitor was also found to improve glucose tolerance in mice lacking the gene to produce GLP-1 receptors. This suggests that DPP4 inhibition is a valid pharmacological approach that improves blood glucose regulation by controlling the activity of GLP-1 as well as additional substrates including a related incretin hormone, Gastric inhibitory Polypeptide (GIP), see, Marguet D, et al., Supra. Other studies have also shown that pharmacological inhibition of DPP4 enzyme activity improves glucose clearance in type 2 diabetic animals, see, Deacon C F, et al. Diabetes 47:764 769 (1998); Pederson R A, et al. Diabetes 47:1253-1258 (1998); Paalg R P, et al. Metab-Clin Exp 48:385-389 (1999); and Balkan B. Diabetologia 42:1324-1331 (1999). These data reveal the value of DPP4 inhibitors in physiological glucose homeostasis and the potential for inhibitors or other modulators of DPP4 activity to be effective treatments for diseases involving altered glucose homeostasis, including diabetes, as well as conditions capable of being modified by the presence, concentration or activity of the enzyme DPP4.

Agents that inhibit or modify the activity of DPP4 are expected to be unique and useful agents to treat diabetes mellitus and other diseases in man. At least one DPP4 inhibitor, i.e., 2-Pyrrolidinecarbonitrile, 1-[[[2-[(5-cyano-2-pyridinyl) amino]ethyl]amino ]acetyl ]-, (2S), has been tested in a multicenter, double-blind, randomized, parallel clinical study, comparing the effect of the inhibitor at various doses with placebo in patients with type 2 diabetes (NIDDM) previously treated with diet only, see Ahren B, et al. Diabetes 50(Suppl 2):A104 (2001)

Syndrome-X

Syndrome-X is a metabolic syndrome that is thought to be related to diabetes. The term syndrome-X was given by Reaven et al describing a condition characterized by central obesity, and metabolic manifestations including resistance to insulin stimulated glucose uptake, hyperinsulinemia, glucose intolerance (not necessarily overt diabetes), increased level of very low density lipoprotein triglyceride (VLDL), decreased level of high density lipoprotein cholesterol (HDL) concentrations and hypertension. Each of these characteristic features are considered to be risk factors for development of atherosclerosis and other ‘old age’ diseases. It is believed that syndrome-X is caused by insulin resistance, but no treatment is available at present. See,. Reaven, G. Diabetes. 37:1595-1607, 1988 and Ferrannini, E. et al. Diabetologia. 34:416-422, 1991.

Developments in Molecular Biology and Genetics

During the past two decades, remarkable developments in molecular biology and genetics have produced a revolutionary growth in understanding of the implication of genes in human disease. Genes have been shown to be directly causative of certain disease states. For example, it has long been known that sickle cell anemia is caused by a single mutation in the human beta globin gene. In many other cases, genes play a role together with environmental factors and/or other genes to either cause disease or increase susceptibility to disease. Prominent examples of such conditions include:
the role of DNA sequence variation in ApoE in Alzheimer's disease,
CKR5 in susceptibility to infection by HIV;
Factor V in risk of deep venous thrombosis;
MTHFR in cardiovascular disease and neural tube defects;
p53 in HPV infection;
various cytochrome p450s in drug metabolism;
and HLA in autoimmune disease.

Surprisingly, the genetic variations that lead to gene involvement in human disease are relatively small. Approximately 1% of the DNA bases which comprise the human genome are polymorphic, that is they are variable between individuals. The genomes of all organisms, including humans, undergo spontaneous mutation in the course of their continuing evolution. The majority of such mutations create polymorphisms, thus the mutated sequence and the initial sequence co-exist in the species population. However, the majority of DNA base differences are functionally inconsequential in that they neither affect the amino acid sequence of encoded proteins nor the expression levels of the encoded proteins. Some polymorphisms that lie within genes or their promoters do have a phenotypic effect and it is this small proportion of the genome's variation that accounts for the genetic component of all difference between individuals, e.g., physical appearance, disease susceptibility, disease resistance, and responsiveness to drug treatments. The relation between human genetic variability and human phenotype is a central theme in modem human genetic studies. The human genome comprises approximately 3 billion bases of DNA.

Single Nucleotide Polymorphisms

Sequence variation in the human genome consists primarily of single nucleotide polymorphisms (“SNPs”) with the remainder of the sequence variations being short tandem repeats (including micro-satellites), long tandem repeats (mini-satellite) and other insertions and deletions. A SNP is a position at which two alternative bases occur at appreciable frequency (i.e. >1%) in the human population. A SNP is said to be “allelic” in that due to the existence of the polymorphism, some members of a species may have the unmutated sequence (i.e., the original “allele”) whereas other members may have a mutated sequence (i.e., the variant or mutant allele). In the simplest case, only one mutated sequence may exist, and the polymorphism is said to be diallelic. The occurrence of alternative mutations can give rise to triallelic polymorphisms, etc. SNPs are widespread throughout the genome and SNPs that after the function of a gene may be direct contributors to phenotypic variation. Due to their prevalence and widespread nature, SNPs have potential to be important tools for locating genes that are involved in human disease conditions, see e.g., Wang et al., Science 280: 1077-1082 (1998), which discloses a pilot study in which 2,227 SNPs were mapped over a 2.3 megabase region of DNA.

An association between a single nucleotide polymorphisms and a particular phenotype does not indicate or require that the SNP is causative of the phenotype. Instead, such an association may indicate only that the SNP is located near the site on the genome where the determining factors for the phenotype exist and therefore is more likely to be found in association with these determining factors and thus with the phenotype of interest. Thus, a SNP may be in linkage disequilibrium (LD) with the ‘true’ functional variant. LD, also known as allelic association exists when alleles at two distinct locations of the genome are more highly associated than expected.

Thus a SNP may serve as a marker that has value by virtue of its proximity to a mutation that causes a particular phenotype.

SNPs that are associated with disease may also have a direct effect on the function of the gene in which they are located. A sequence variant may result in an amino acid change or may alter exon-intron splicing, thereby directly modifying the relevant protein, or it may exist in a regulatory region, altering the cycle of expression or the stability of the mRNA, see Nowotny P Current Opinions in Neuobiology, 2001, 11:637-641.

The role that a common genomic variant might play in susceptibility to disease is best exemplified by the role that the apolipoprotein E (APOE) ε4 allele plays in Alzheimer's disease (AD). The ε4 allele is highly associated with the presence of AD and with earlier age of onset of disease. It is a robust association seen in many populations studied, see St George-Hyslop et al. Biol Psychiatry 2000, 47:183-199. Polymorphic variation has also been implicated in stroke and cardiovascular disease, see Wu et al. Am J Cardiol 2001, 87; 1361-1366 and in multiple sclerosis, see Oksenberg et al. J Neuroimmuol 2001, 113:171-184.

It is increasingly clear that the risk of developing many common disorders and the metabolism of medications used to treat these conditions are substantially influenced by underlying genomic variations, although the effects of any one variant might be small.

Therefore, an association between a SNP and a clinical phenotype suggests, 1) the SNP is functionally responsible for the phenotype or, 2) there are other mutations near the location of the SNP on the genome that cause the phenotype. The 2^{nd} possibility is based on the biology of inheritance. Large pieces of DNA are inherited and markers in close proximity to each other may not have been recombined in individuals that are unrelated for many generations, i.e., the markers are in linkage disequlibrium (LD).

The available evidence strongly suggests that compounds or therapies that modify or inhibit DPP4 activity or otherwise act to improve metabolic or glycemic control in patients with disorders of impaired glycemic control will be useful in the treatment of disorders characterized by impaired glycemic control such as diabetes and other related diseases. These compounds or agents include but are not limited to the DPP4 inhibitors, 2-Pyrrolidinecarbonitrile, 1-[[[2-[(5-cyano-2-pyridinyl) amino]ethyl]amino]acetyl]-, (2S) and (1-[3Hydroxy-adamant-1-ylamino)-acetyl]-pyrrolidine-2(S)-carbonitrile).

However, in the past, there has been no way to determine which individuals will respond to DPP4 modifiers or other glycemic control agents and which will not. Thus, there is a need for methods to determine those individuals who suffer from impaired glycemic control, who will respond to glycemic control agents or therapies, including but not limited to a DPP4 modifiers or inhibitors or other anti-diabetic agents, or to any agent or therapy intended to improve glycemic control, and those who will not. In addition, there is a need for methods to determine those individuals, with impaired glycemic control who will respond to low dose treatment and those individuals who will require higher doses to obtain optimal results and therefore custom tailor the treatment to the individual to provide effective treatment with minimal side effects and danger of drug interaction. In addition, there is a need for methods to optimize clinical trials of glycemic control agents or therapies to take into account the significant variation in response that these individuals are now known to have.

### SUMMARY OF THE INVENTION

The present invention, as described herein below, overcomes deficiencies in currently available methods to treat diabetes with glycemic control agents or therapies, such as DPP4 modifiers or inhibitors, by identifying a polymorphism in the TCF1 locus which is associated with the clinical response to a glycemic control agent or therapy, such as a DPP4 modifier or inhibitor, including but not limited to 2-Pyrrolidinecarbonitrile, 1-[[[2-[(5-cyano2-pyridinyl) amino]ethyl]amino]acetyl]-, (2S) and 1-[3Hydroxy-adamant-1-ylamino)-acetyl]pyrrolidine-2(S)-carbonitrile. The identification of this polymorphism allows the development of a simple test to determine which patients will respond to DPP4 modifier or inhibitor therapy, including therapy with 2-Pyrrolidinecarbonitrile, 1-[[[2-[(5-cyano-2-pyridinyl) amino]ethyl]amino]acetyl]-, (2S), or 1-[3Hydroxy-adamant-1-ylamino)-acetyl]-pyrrolidine-2(S)-carbonitrile or other GLP-1 based therapies, and therapies acting through other mechanisms of action that tend to normalize glycemic control, and to predict required dosage levels. This will allow the clinician to make a more informed decision about whether or not to treat a patient with diabetes with a glycemic control agent or therapy such as a DPP4 modifier or inhibitor and, if so, how much to use.

These agents and therapies include, but are not limited to, GLP-1 or analogs thereof including synthetic analogs or natural mimetics, including Exendin-4, and agents activating the GLP-1 receptor, agents activating receptors for GIP, PACAP, or glucagon, drugs affecting insulin secretion or glucose sensing by pancreatic beta cells, including sulfonylurea agents, meglitinide agents, agents affecting glucokinase activity, agents affecting phosphodiesterase activity, agents affecting glucose production or intermediary metabolism including inhibitors of glucagon secretion or action, modulators of glucocorticoid receptor activation, biguanides, inhibitors of acetyl CoA carboxylase and other activators of fatty acid oxidation, therapies affecting insulin action, including compounds activating or modulating the PPAR family of nuclear hormone receptors, inhibitors of protein phosphatases, inhibitors of glycogen synthase kinase, inhibitors of the NFkB pathway, SHP2 modulators, insulin mimetic agents and biguanides and including therapies affecting energy balance, including inhibitors of dietary fat digestion or absorption (pancreatic lipase, fatty add transport protein, microsomal triglyceride transfer protein, bile acid transporter, diacylglyceride acyltransferase, or pancreatic proteinase inhibitors, and, in addition, therapies affecting carbohydrate digestion, glucose absorption or intestinal glucose utilization, including inhibitors of alpha-glucosidase, inhibitors of amylase and agents delaying gastric emptying such as amylin, or biguanides

Therefore, the present invention provides methods to make use of the TCF-1 genotype of an individual in assessing the utility of glycemic control agents or therapies, including DPP4 inhibitors in the management of diseases characterized by impaired glycemic control, including: type 2 diabetes, type 1 diabetes, impaired glucose tolerance, impaired fasting glucose, Syndrome X, prandial lipemia, hypercholesterolemia, impaired glucose metabolism, gestational diabetes, and abnormal prandial glycemic response (PGR) refering to an excessive or abnormal increase in serum glucose during the prandial period (prandial or post-prandial hyperglycemia).

Thus the present invention provides methods for determining the responsiveness of an Individual with a disorder characterized by impaired glycemic control to treatment with a glycemic control agent or therapy, comprising; determining for the two copies of the TCF1 gene present in the individual, the identity of the nucleotide pair at the polymorphic site at 483 A>G, and assigning the individual to a good responder group if both pairs are GC or if one pair is AT and one pair is GC and to a low responder group if both pairs are AT.

The method may make use of any glycemic control agents or therapies including, but not limited to, a dipeptidylpeptidase 4 (DPP4) inhibitor such as 2-Pyrrolidinecarbonitrile, 1-[[[2-[(5cyano-2-pyridinyl) amino]ethyl]amino]acetyl]-, (2S) or 1-[3Hydroxy-adamant-1-ylamino)-acetyl]-pyrrolidine-2(S)-carbonitrile or any of the compounds of Formula I or Formula II.

The methods may be used to treat any disorder characterized by impaired glycemic control including, but not limited to; type 2 diabetes mellitus, type 1 diabetes mellitus, impaired glucose tolerance, impaired fasting glucose, Syndrome X, gestational diabetes or any disorder responsive to DPP4 inhibitors

In another embodiment the present invention provides methods for treating an individual with a disorder characterized by impaired glycemic control comprising, determining for the two copies of the TCF1 gene present in the individual, the identity of the nucleotide pair at the polymorphic site 483 A>G, wherein, if both the nucleotide pairs are CG or if one is AT and one is CG the individual is treated with a glycemic control agent or therapy and if the nucleotide pairs are both AT the individual is treated with alternate therapy.

These methods may make use of any glycemic control agents or therapies including but not limited to; a dipeptidylpeptidase 4 (DPP4) inhibitor such as 2-Pyrrolidinecarbonitrile, 1-[[[2-[(5-cyano-2-pyridinyl) amino]ethyl]amino]acetyl]-, (2S) or 1-[(3Hydroxy-adamant-1-ylamino)-acetyl]-pyrrolidine-2(S)-carbonitrile or any of the compounds of Formula I or Formula II.

These methods nay be used to treat any disorder characterized by impaired glycemic control including, but not limited to, type 2 diabetes mellitus, type 1 diabetes mellitus, impaired glucose tolerance, impaired fasting glucose, Syndrome X, gestational diabetes or any disorder responsive to DPP4 inhibitors

In a further embodiment the present invention provides methods for identifying an association between a trait and at least one genotype or haplotype of the TCF1 gene which comprises, comparing the frequency of the genotype or haplotype in a population exhibiting the trait with the frequency of the genotype or haplotype in a reference population, wherein the genotype or haplotype comprises a nucleotide pair or nucleotide located at the polymorphic site 483 A>G, wherein a higher frequency of the genotype or haplotype in the trait population than in the reference population indicates the trait is associated with the genotype or haplotype. This trait may be, but is not limited to, a clinical response to a drug targeting TCF1 or DPP4.

In a further embodiment the present invention provides methods for treating an individual, with a disorder characterized by impaired glycemic control, the method comprising, determining for the two copies of the TCF1 gene present in the individual, the identity of the nucleotide pair at the polymorphic site 483 A>G, wherein, if both the nucleotide pairs are CG or if one is AT and one is CG the individual is treated with a low dose of a glycemic control agent and if the nucleotide pairs are both AT the individual is treated with a high dose of a glycemic control agent.

The above method may make use of any glycemic control agents or therapies including but not limited to, a dipeptidylpeptidase 4 (DPP4) inhibitor such as 2-Pyrrolidinecarbonitrile, 1-[[[2-[(5-cyano-2-pyridinyl) amino]ethyl]amino]acetyl]-, (2S) or 1-[3Hydroxy-adamant-1-ylamino)-acetyl]-pyrrolidine-2(S)-carbonitrile or any of the compounds of Formula I or Formula II.

The above methods may be used to treat any disorder characterized by impaired glycemic control including, but not limited to; type 2 diabetes mellitus, type 1 diabetes mellitus, impaired glucose tolerance, impaired fasting glucose, Syndrome X, gestational diabetes or any disorder responsive to DPP4 inhibitors

In a further embodiment, the present invention provides a method of treating a patient with a disorder characterized by impaired glycemic control comprising, providing genetic counseling to the patient and patients family, determining the patients genotype for the TCF1 gene at the polymorphism site 483 A>G, and then determining the proper therapy for said patient based on results of the genotype determination.

In a further embodiment the present invention provides a method for optimizing clinical trial design for glycemic control agents, comprising, determining, for the two copies of the TCF1 gene present in an individual being considered for inclusion in the clinical trial, the identity of the nucleotide pair at the polymorphic site 483 A>G, wherein, if both the nucleotide pairs are CG or if one is AT and one is CG the individual is included in the clinical trial and if the nucleotide pairs are both AT the individual is not included.

In a further embodiment the present invention provides a method for identifying individuals, with a disorder characterized by impaired glycemic control, who would benefit from drug A vs. B, comprising, determining, for the two copies of the TCF1 gene present in the individual, the identity of the nucleotide pair at the polymorphic site 483 A>G, wherein, if both the nucleotide pairs are CG or if one is AT and one is CG the individual would benefit from a glycemic control agent or therapy and if the nucleotide pairs are both AT the individual would benefit from an alternate glycemic control agent or therapy.

In a further embodiment the present invention provides a method for determining which individuals, with a disorder characterized by impaired glycemic control, could be treated with a glycemic control agents with reduced side effects, comprising, determining, for the two copies of the TCF1 gene present in the individual, the identity of the nucleotide pair at the polymorphic site 483 A>G, wherein, if both the nucleotide pairs are CG or if one is AT and one is CG the individual can be treated with lower doses of a glycemic control agent with fewer side effects and if the nucleotide pairs are both AT the individual must be treated with higher doses of a glycemic control agent and therefore greater side effects.

In a further embodiment, the invention provides methods for determining the responsiveness of an individual with a disorder characterized by impaired glycemic control to treatment with a glycemic control agent or therapy, comprising; determining, for the two copies of the TCF1 gene present in the individual, the identity of a nucleotide pair at a polymorphic site in the region of the TCF1 gene that is in linkage disequilibrium with the polymorphic site at TCF1 483 A>G, and assigning the individual to a good responder group if the nucleotide pair at a polymorphic site in the region of the TCF1 gene that is in linkage disequilibrium with the polymorphic site at 483 A>G, indicates that, at the TCF1 polymorphic site at 483 A>G, both nucleotide pairs are GC or one pair is AT and one pair is GC and to a low responder group if said nucleotide pair indicates that both pairs are AT at the TCF1 483 A>G site.

### BRIEF DISCUSSION OF THE DRAWING

FIG. 1 is a diagram showing the mean (±SEM) prandial glycemic response for each of the alleles of TCF1 for the polymorphism at 483 A>G, i.e., AG, AA and GG, for subjects treated with placebo or with a DPP-IV inhibitor as described in the text. Levels of significant differences between placebo and inhibitor-treated subjects of the same genotype are indicated within the figure.

FIG. 2 is a diagram showing the mean (±SEM) glycosylated hemoglobin (HbA1c) response for each of the alleles of TCF1 for the polymorphism at 483 A>G. i.e., AG, AA and GG for subjects treated with placebo or with a DPP-IV inhibitor as described in the text. Levels of significant differences between placebo and inhibitor-treated subjects of the same genotype are indicated within the figure.

FIG. 3 Shows the sequence of the section of the TCF1 gene where the 483 A>G polymorphism is located (SEQ ID NO: 1). This sequence is derived from GenBank accession number U72616. The polymorphic nucleotide is located at nucleotide No, 183 in SEQ ID NO: 1, and may be A or G. Also indicated in this sequence in FIG. 3 are the sequences used for the forward and reverse primers used for PCR amplification. SEQ ID NO: 2 is the invader probe and Probe 1 and Probe 2 are SEQ ID NOS: 3 and 4 respectively. In FIG. 3 the nucleotide marked with * is the nucleotide that is polymorphic, the nucleotides in bold represent the forward and reverse primers used for PCR amplification and the underlined nucleotides represent the extension primers.

### DESCRIPTION OF THE PREFERRED EMBODIMENTS

The DPP4 Inhibitor Study

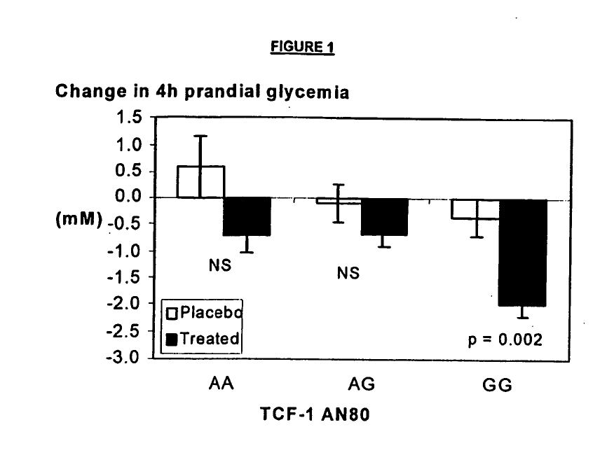

The genotypes of 76 individuals, enrolled in a study of a specific inhibitor of DPP4 in diabetic patients, were examined for polymorphisms in 91 loci in an effort to identify genetic determinants (such as SNPs) or correlates of response to the DPP4 inhibitor being studied, i.e., 2-Pyrrolidinecarbonitrile, 1-[[[2-[(5-cyano-2-pyridinyl) amino]ethyl]amino]acetyl]-(2S). The genetic loci examined included those genes thought to be related to the pathway of the anti-diabetic action of the compound as well as genes thought to be related to the genetic etiology of diabetes. A highly significant relationship (p=0.00051) was found between the 48 A>G polymorphism at the TCF1 locus and the treatment response in the integrated exposure to glucose measured during a four hour standardized breakfast meal. This response is referred to as the prandial glycemic response (PGR), see FIG. 1.

The product of the TCF1 gene is TCF1 transcription factor 1, hepatic. This transcription factor is also known as; LF-B1, hepatic nuclear factor-1 alpha (HNF-1 alpha) and albumin proximal factor and is known to regulate the activation of genes responsible for insulin response. Mutations in the TCF1 gene have been previously associated with susceptibility to MODY type 3, See, Urhammer S A, Diabetologia 1997, 40(4):473-5.

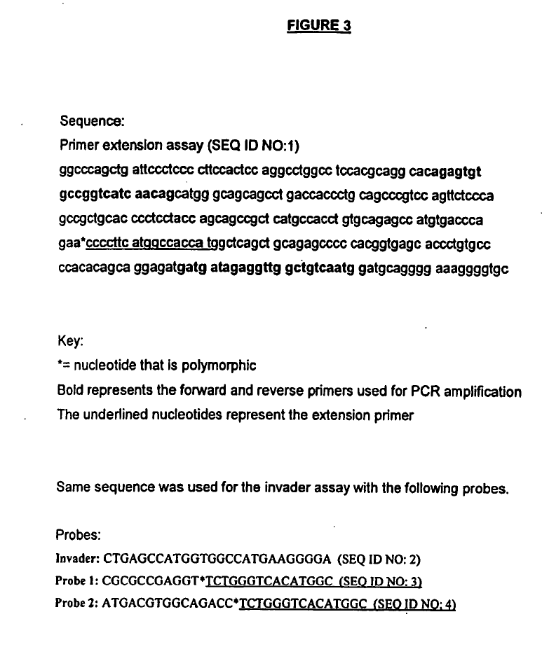

The TCF1 gene is located at chromosome location: 12q24.2. The standard nomenclature for the nucleotide substitution for the polymorphism of this invention is 483 A>G and consequent amino acid substitution in the expressed polypeptide product is Asn 487 Ser. This polymorphism was reported in 1997, See, Urhammer S A, Diabetologia 1997, 40(4):473-5 (PMID: 9112026). The polymorphism is located in the partial sequence shown in FIG. 3, and was derived from GenBank accession number U72616.

Among the DPP4 treated individuals there was a significant difference in the prandial glycemic response (PGR) between individuals of the GG genotype and individuals with the AG or AA genotype with GG homogenous patients having the best response to 2-Pyrrolidinecarbonitrile, 1-[[[2[(5cyano-2-pyridinyl) amino]ethyl]amino]acetyl]-, (2S) in the sense of improved glucose homeostasis with treatment.

It is now recognized that prandial glycemic control is one element of an integrated strategy to reduce complications of diabetes that are thought to be driven by the combined increase in glucose exposure during the prandial period as well as from elevated fasting plasma glucose concentrations. Any strategy to improve the impact of a given agent on the overall glycemic control must take into account the need to improve this integrated exposure.

As used herein, the term “prandial” shall mean during the meal.

As used herein, the term “post-prandial” shall mean during the absorbtion period following meal intake (approximatly 0-8 hours, depending on the meal sixe and composition).

As used herein, the term “post-absorptive” shall mean after nutrient absorption is completed or approximatly 4-8 hours post-meal.

As used herein, the term “fasting” shall mean after a prolonged period i.e. 12-16 hours, without eating.

As used herein, the term “prandial glycemic response” (PGR) refers to the change in serum glucose during the prandial or post-prandial period.

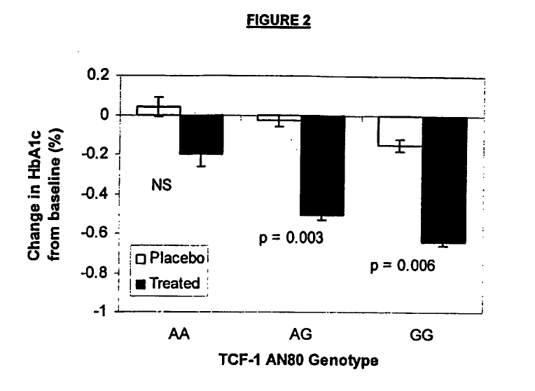

The level of glycosylated hemoglobin (HbA1c) in circulating erythrocytes has been firmly established as an integrated marker of glycemic control that reflects long-term exposure to glucose concentrations. In the present invention, it has been discovered that in addition to the relationship between prandial glycemic response and the GG TCF1 genotype, both TCF1 AG and TCF1 GG genotypes are associated with an overall improvement in glycemic control, evidenced by an association of the AG and GG TCF1 genotypes with improved changes in glycosylated hemoglobin (HbA1c) levels after four weeks of treatment with 2-Pyrrolidinecarbonitrile, 1-[[[2-[(5-cyano-2-pyridinyl) amino]ethyl]amino]acetyl]-, (2S) (see FIG. 2).

As used herein the term “disorders characterized by impaired glycemic control” (IGC) shall mean a metabolic disorder in which one of the primary manifestation is the excessive or abnormal elevation of blood glucose levels, either in the fasting state or in response to a meal or an oral glucose load and shall include; type 2 diabetes, type 1 diabetes, impaired glucose metabolism. i.e., impaired glucose tolerance (post-prandial hyperglycemia) and/or impaired fasting glucose, Syndrome X, gestational diabetes and abnormal prandial glycemic response (PGR refering to an excessive or abnormal increase in serum glucose during the prandial period (prandial or post-prandial hyperglycemia).

As used herein, the term “glycemic control agent or therapy” shall mean any compound, drug or form of treatment that, in a patient with; type 2 diabetes or type 1 diabetes, impaired glucose tolerance, impaired fasting glucose, Syndrome X, post-prandial hyperglycemia or gestational diabetes will tend to normalize fasting, prandial or post-prandial serum glucose levels or to normalize glycosylated hemoglobin (HbA1c) response over time.

The term “DPP4 inhibitor”, as used herein, means a compound capable of inhibiting the catalytic actions of the enzyme DPP4 (DPP-IV; dipeptidylpeptidase IV; EC 3.4.14.5), which is a serine exopeptidase identical to ADA complexing protein-2 and to the T-cell activation antigen CD26.

Many compounds that act as inhibitors of DPP4 enzyme activity are now known, such as 2-Pyrrolidinecarbonitrile, 1-[[[2-[(5-cyano-2-pyridinyl) amino]ethyl]amino]acetyl]-, (2S) and (1-[3Hydroxy-adamant-1-ylamino)-acetyl]-pyrrolidine-2(S)-carbonitrile) and including, but not limited to, the compounds disclosed in U.S. Pat. Nos.; 6,011,155, 6,124,305, 6,166,063, 5,602,102, 6,110,949, 6,274,608 B1, 5,462,928, 6,172,081, 6,107,317, 6,110,949, 6,172,081, 5,939,560, 5,543,396, and 6,107,317 and international Publications WO 01/34594 A1, WO 01/47514 A1, WO 00/34241, WO 01/55085 A1, WO 01/52825 A2, WO 01/04156 A1, WO 00/10549, WO 01/55105 A1, WO 99/67278, WO 95/15309, WO 98/19998, WO 01/34594, WO 01/62266, WO 97/40832, WO 01/72290, WO 01/68603, WO 00/34241, WO 99/61431, WO 99/67279, WO 93/08259, WO 95/11689, WO 91/16339, WO 93/08259, WO 95/11689, WO 95/29691, WO 95/34538, WO 99/46272, WO 95/29691, WO 00/53171 and WO 99/38501 and EP1052994, EP1019494, EP0528858, EP0610317, EP1050540, EP1062222 and German Patents Nos. 158109 and 296075, the contents of all of these patents and publications are hereby incorporated by reference herein for all purposes. Any of the DPP4 inhibitors disclosed in the above patents and publications may be used in the methods of the present invention. Particularly preferred DPP4 inhibitors are the compounds 2-Pyrrolidinecarbonitrile, 1-[[[2-[(5-cyano-2-pyridinyl) amino]ethyl]amino]acetyl]-, (2S) and (1-[3Hydroxy-adamant-1-ylamino)-acetyl-pyrrolidine-2(S)-carbonitrile).

Therefore, the present invention is based, in part, on the discovery of the novel association, in patients with disorders characterized by impaired glycemic control, of genetic variants or single nucleotide polymorphisms (“SNPs”) of the TCF1 gene with the clinical response to glycemic control agents or therapies including but not limited to administration of a DPP4 inhibitor.

As described in detail below, these variants are associated with significant variation in the clinical response to modifiers or inhibitors of the enzyme DPP4 in the treatment of diabetes and other diseases that are responsive to inhibitors or modifiers of the activity of the enzyme DPP4, including therapy with 2-Pyrrolidinecarbonitrile, 1-[[[2-[(5-cyano-2-pyridinyl) amino]ethyl]amino]acetyl]-, (2S), and other GLP-1 based therapies, and therapies acting through other similar mechanisms of action that tend to stabilize glycemic control. These variants were found in genomic DNAs isolated from 76 individuals participating in a study of the effect of the DPP4 inhibitor, 2-Pyrrolidinecarbonitrile, 1-[[[2-[(5-cyano-2-pyridinyl) amino]ethyl]amino]acetyl]-, (2S), in the treatment of type 2 diabetes (NIDDM).

Formula I Compounds

Other DPP4 inhibitors that may be used in the present invention include, but are not limited to, the following N-(N′-substituted glycyl)-2-cyanopyrrolidines, these, as a group constitute formula I as described below; 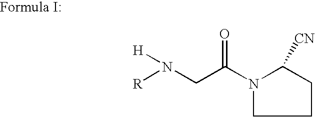
wherein R is:
a) R₁R₁ₐN(CH₂)ₘ— wherein
R₁ is a pyridinyl or pyrimidinyl moiety optionally mono- or independently disubstituted with (C₁₋₄)alkyl, (C₁₋₄)alkoxy, halogen, trifluoromethyl, cyano or nitro; or phenyl optionally mono- or independently disubstituted with (C₁₋₄)alkyl, (C₁₋₄)alkoxy or halogen; R₁ₐ is hydrogen or (C₁₋₈)alkyl; and m is 2 or 3;
b) (C₃₋₁₇)cycloalkyl optionally monosubstituted in the 1-position with (C₁₋₃)hydroxyalkyl;
c) R₂(CH₂)ₙ— wherein either
R₂ is phenyl optionally mono- or independently di- or independently trisubstituted with (C₁₋₄)alkyl, (C₁₋₄)alkoxy, halogen or phenylthio optionally monosubstituted in the phenyl ring with hydroxymethyl; or is (C₁₋₈)alkyl; a [3.1.1]bicyclic carbocyclic moiety optionally mono- or plurisubstituted with (C₁₋₈)alkyl; a pyridinyl or naphthyl moiety optionally mono- or independently disubstituted with (C₁₋₄)alkyl, (C₁₋₄)alkoxy or halogen; cyclohexene; or adamantyl; and
n is 1 to 3; or
R₂ is phenoxy optionally mono- or independently disubstituted with (C₁₋₄)alkyl, (C₁₋₄)alkoxy or halogen; and
n is 1 or 3;
d) (R₃)₂CH(CH₂)₂— wherein each R₃ independently is phenyl optionally mono- or independently disubstituted with (C₁₋₄)alkyl, (C₁₋₄)alkoxy or halogen;
e) R₄(CH₂)ₚ— wherein R₄ is 2-oxopyrrolidinyl or (C₂₋₄)alkoxy and p is 2 to 4;
f) isopropyl optionally monosubstituted in 1-position with (C₁₋₃)hydroxyalkyl;
g) R₅ wherein R₅ is: indanyl; a pyrrolidinyl or piperidinyl moiety optionally substituted with benzyl; a [2.2.1]- or [3.1.1]bicyclic carbocyclic moiety optionally mono- or plurisubstituted with (C₁₋₈)alkyl; adamantyl; or (C₁₋₈)alkyl optionally mono- or independently plurisubstituted with hydroxy, hydroxymethyl or phenyl optionally mono- or independently disubstituted with (C₁₋₄)alkyl, (C₁₋₄)alkoxy or halogen;
   in free form or in acid addition salt form.

The compounds of formula I can exist in free form or in acid addition salt form. Salt forms may be recovered from the free form in known manner and vice-versa. Acid addition salts may, e.g., be those of pharmaceutically acceptable organic or inorganic acids. Although the preferred acid addition salts are the hydrochlorides, salts of methanesulfonic, sulfuric, phosphoric, citric, lactic and acetic add may also be utilized.

The compounds of formula 1 may exist in the form of optically active isomers or diastereoisomers and can be separated and recovered by conventional techniques, such as chromatography.

“Alkyl” and “alkoxy” are either straight or branched chain, of which examples of the latter are isopropyl and tert-butyl.

R preferably is a), b) or e) as defined above. R₁ preferably is a pyridinyl or pyrimidinyl moiety optionally substituted as defined above. R₁a preferably is hydrogen. R₁ₐ preferably is phenyl optionally substituted as defined above. R₃ preferably is unsubstituted phenyl. R₄ preferably is alkoxy as defined above. R₅ preferably is optionally substituted alkyl as defined above. m preferably is 2. n preferably is 1 or 2, especially 2. p preferably is 2 or 3, especially 3.

Pyridinyl preferably is pyridin-2-yl; it preferably is unsubstituted or monosubstituted, preferably in 5-position. Pyrimidinyl preferably is pyrimidin-2-yl. It preferably is unsubstituted or monosubstituted, preferably in 4-position. Preferred as substitutents for pyridinyl and pyrimidinyl are halogen, cyano and nitro, especially chlorine.

When it is substituted, phenyl preferably is monosubstituted; it preferably is substituted with halogen, preferably chlorine, or methoxy. It preferably is substituted in 2-, 4- and/or 5-position, especially in 4-position. (C₃₋₁₂) cycloalkyl preferably is cyclopentyl or cyclohexyl. When it is substituted, it preferably is substituted with hydroxymethyl. (C₁₋₄) alkoxy preferably is of 1 or 2 carbon atoms, it especially is methoxy. (C₂₋₄) alkoxy preferably is of 3 carbon atoms, it especially is isopropoxy. Halogen is fluorine, chlorine, bromine or iodine, preferably fluorine, chlorine or bromine, especially chlorine. (C₁₋₄) alkyl preferably is of 1 to 6, preferably 1 to 4 or 3 to 5, especially of 2 or 3 carbon atoms, or methyl. (C₁₋₄) alkyl preferably is methyl or ethyl, especially methyl. (C₁₋₄) hydroxyalkyl preferably is hydroxymethyl.

A [3.1.1]bicyclic carbocyclic moiety optionally substituted as defined above preferably is bicyclo[3.1.1]hept-2-yl optionally disubstituted in 6-position with methyl, or bicyclo[3.1.1]hept-3-yl optionally trisubstituted with one methyl in 2-position and two methyl groups in 6-position. A [2.2.1]bicyclic carbocyclic moiety optionally substituted as defined above preferably is bicyclo[2.2.1]hept-2-yl.

Naphthyl preferably is 1-naphthyl. Cyclohexene preferably is cyclohex-1-en-1-yl. Adamantyl preferably is 1- or 2-adamantyl.

A pyrrolidinyl or piperidinyl moiety optionally substituted as defined above preferably is pyrrolidin-3-yl or piperidin-4yl. When it is substituted it preferably is N-substituted.

A preferred group of compounds of formula 1 are the compounds wherein R is R′ (compounds Ia), whereby R′ is: R₁′NH(CH₂)₂— wherein R₁′ is pyridinyl optionally mono- or independently disubstituted with halogen, trifluoromethyl, cyano or nitro; or unsubstituted pyrimidinyl; (C₃₋₇)cycloalkyl optionally monosubstituted in 1-position with (C₁₋₃)hydroxyalkyl; R₄′(CH₂)₃— wherein R₄′ is (C₂₋₄)alkoxy; or R₅, wherein R₅ is as defined above; in free form or in acid addition salt form.

More preferred compounds of formula I are those wherein R is R′ (compounds Ib), whereby R″ is: R₁″NH(CH₂)₂— wherein R₁″ is pyridinyl mono- or independently disubstituted with halogen, trifluoromethyl, cyano or nitro; (C₄₋₈)cycloalkyl monosubstituted in 1-position with (C₁₋₃)hydroxyalkyl; R₄′(CH₂)₃— wherein R₄′ is as defined above; or R₅′ wherein R₅′ is a [2.2.1]- or [3.1.1]bicyclic carbocyclic moiety optionally mono- or plurisubstituted with (C₁₋₈)alkyl; or adamantyl; in free form or in acid addition salt form.

Even more preferred compounds of formula I are those wherein R is R′″ (compounds Ic), whereby R′″ is: R₁″NH(CH₂)₂— wherein R₁″ is as defined above; (C₁₋₈)cycloalkyl monosubstituted in 1-position with hydroxymethyl; R₄′(CH₂)₃— wherein R₄′ is as defined above; or R₅″ wherein R₅″ is adamantyl; in free form or in acid addition salt form.

A further group of compounds are Ip, wherein R is R^{p}, which is:
a) R₁^{p}NH(CH₂)₂— wherein R₁^{p}is a pyridinyl or pyrimidinyl moiety optionally mono- or independently disubstituted with halogen, trifluoromethyl, cyano or nitro;
b) (C₃₋₇)cycloalkyl optionally monosubstituted in 1-position with (C₁₋₄)hydroxyalkyl;
c) R₁^{p}(CH₂)₂— wherein R₂^{p} is phenyl optionally mono- or independently di- or independently trisubstituted with halogen or (C₁₋₃)alkoxy;
d) (R₃^{p})₂CH(CH₂)₂— wherein each R₃^{p} independently is phenyl optionally monosubstituted with halogen or (C₁₋₃)alkoxy;
e) R₄(CH₂)₃— wherein R₄ is as defined above; or
f) isopropyl optionally monosubstituted in 1-position with (C₁₋₃)hydroxyalkyl; in free form or in pharmaceutically acceptable acid addition salt form.

A further group of compounds are those wherein R is R^{s}, which is:
a) R₁^{s}R₁ₐ^{s}(CH₂)ₘₛ— wherein R₁^{s} is pyridinyl optionally mono- or independently disubstituted with chlorine, trifluoromethyl, cyano or nitro; pyrimidinyl optionally monosubstituted with chlorine or trifluoromethyl; or phenyl; R₁ₐ^{s} is hydrogen or methyl; and ms is 2 or 3;
b) (C₃₋₁₂)cycloalkyl optionally monosubstituted in 1-position with hydroxymethyl;
c) R₂^{s}(CH₂)ₘₛ— wherein either R₂^{s} is phenyl optionally mono- or independently di- or independently trisubstituted with halogen, alkoxy of 1 or 2 carbon atoms or phenylthio monosubstituted in the phenyl ring with hydroxymethyl; (C₁₋₆)alkyl; 6,6-dimethylbicyclo[3.1.1]hept-2-yl; pyridinyl; naphthyl; cyclohexene; or adamantyl; and ns is 1 to 3; or R₂^{s} is phenoxy; and ns is 2;
d) (3,3-diphenyl)propyl;
e) R₄^{s}(CH₂)ₚₛ wherein R₄^{s} is 2-oxopyrrolidin-1-yl or isopropoxy and ps is 2 or 3;
f) isopropyl optionally monosubstituted in 1-position with hydroxymethyl;
g) R₅^{s} wherein R₅^{s} is: indanyl; a pyrrolidinyl or piperidinyl moiety optionally N-substituted with benzyl; bicyclo[2.2.1]hept-2-yl; 2,6,6trimethylbicyclo-[3.1.1]hept-3-yl; adamantyl; or (C₁₋₈)alkyl optionally mono- or independently disubstituted with hydroxy, hydroxymethyl or phenyl;
   in free form or in acid addition salt form.
   Formula II Compounds

In addition, other DPP4 inhibitors may be used in the present invention including, but not limited to, the following N-(substituted glycyl)-2- cyanopyrrolidines, these compounds, as a group constitute formula II as described below; 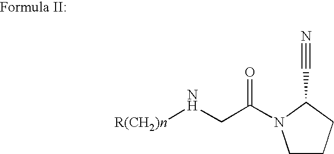
wherein R is substituted adamantyl; and n is 0 to 3; in free form or in acid addition salt form. The compounds of formula II can exist in free form or in acid addition salt form. Pharmaceutically acceptable (i.e., non-toxic, physiologically acceptable) salts are preferred, although other salts are also useful, e.g., in isolating or purifying the compounds of this invention. Although the preferred acid addition salts are the hydrochlorides, salts of methanesulfonic, sulfuric, phosphoric, citric, lactic and acetic acid may also be utilized.

The compounds of the invention may exist in the form of optically active isomers or diastereoisomers and can be separated and recovered by conventional techniques, such as chromatography.

Listed below are definitions of various terms used to describe this invention. These definitions apply to the terms as they are used throughout this specification, unless otherwise limited in specific instances, either individually or as part of a larger group. The term “alkyl” refers to straight or branched chain hydrocarbon groups having 1 to 10 carbon atoms, preferably 1 to 7 carbon atoms, most preferably 1 to 5 carbon atoms. Exemplary alkyl groups include methyl, ethyl, propyl, isopropyl, n-butyl, t-butyl, isobutyl, pentyl, hexyl and the like. The term “alkanoyl” refers to alkyl-C(O)—. The term “substituted adamantyl” refers to adamantyl, i.e., 1- or 2-adamantyl, substituted by one or more, for example two, substitutents selected from alkyl, —OR.sub.1 or —NR.sub.2 R.sub.3; where R.sub.1, R. sub.2 and R.sub.3 are independently hydrogen, alkyl, (C.sub.1-C.sub.8-alkanoyl), carbamyl, or —CO—NR.sub.4 R.sub.5; where R.sub.4 and R.sub. 5 are independently alkyl, unsubstituted or substituted aryl and where one of R.sub.4 and R.sub.5 additionally is hydrogen or R.sub.4 and R.sub. 5 together represent C.sub.2-C.sub.7 alkylene. The term “aryl” preferably represents phenyl. Substituted phenyl preferably is phenyl substituted by one or more, e.g., two, substitutents selected from, e.g., alkyl, alkoxy, halogen and trifluoromethyl. The term “alkoxy” refers to alkyl-O—. The term “halogen” or “halo” refers to fluorine, chlorine, bromine and iodine. The term “alkylene” refers to a straight chain bridge of 2 to 7 carbon atoms, preferably of 3 to 6 carbon atoms, most preferably 5 carbon atoms.

A preferred group of compounds of the invention is the compounds of formula I wherein the substituent on the adamantyl is bonded on a bridgehead or a methylene adjacent to a bridgehead. Compounds of formula II wherein the glycyl-2-cyanopyrrolidine moiety is bonded to a bridgehead, the R′ substituent on the adamantyl is preferably 3-hydroxy. Compounds of formula II wherein the the glycyl-2-cyanopyrrolidine moiety is bonded at a methylene adjacent to a bridgehead, the R′ substituent on the adamantyl is preferably 5-hydroxy.

Particularly preferred DPP4 inhibitors are the compounds; 2-Pyrrolidinecarbonitrile, 1-[[[2[(5-cyano-2-pyridinyl) amino]ethyl]amino]acetyl]-, (2S) and 1-(3Hydroxy-adamant-1-ylamino)-acetyl]-pyrrolidine-2(S)-carbonitrile.

Thus, in a first aspect, the invention provides methods of determining the responsiveness of an individual with; type 2 diabetes, impaired glucose tolerance, impaired fasting glucose, Syndrome X, prandial lipemia, hypercholesterolemia, hypertension, gestational diabetes or type 1 diabetes or any DPP4 inhibitor responsive disorder, to treatment with a DPP4 inhibitor compound or to glycemic control agents or therapies. These methods comprise determining the genotype or haplotype of the TCF1 gene and making the determination of responsiveness based on the presence or absence of one or more polymorphisms in the TCF1 gene. This aspect of the invention also provides methods of determining the responsiveness of an individual with diabetes or a related metabolic disorder, to treatment with other agents or therapies intended to improve metabolic control. The detection of these polymorphisms can be used to determine or predict the responsiveness of the individual to a particular drug or other therapy. One of skill in the art will readily recognize that, in addition to the specific polymorphisms disclosed herein, any polymorphism that is in linkage disequilibrium with the said polymorphism can also serve as a surrogate marker indicating responsiveness to the same drug or therapy as does the SNP that it is in linkage disequilibrium with. Therefore, any SNP in linkage disequilibrium with the SNPs disclosed in this specification, can be used and is intended to be included in the methods of this invention.

Identification and Characterization of SNPs

Many different techniques can be used to identify and characterize SNPs, including single-strand conformation polymorphism analysis, heteroduplex analysis by denaturing high-performance liquid chromatography (DHPLC), direct DNA sequencing and computational methods, see Shi M M, Clin Chem 2001, 47:164-172. Thanks to the wealth of sequence information in public databases, computational tools can be used to identify SNPs in silico by aligning independently submitted sequences for a given gene (either cDNA or genomic sequences). Comparison of SNPs obtained experimentally and by in silico methods showed that 55% of candidate SNPs found by SNPFinder(http://lpgws.nci.nih.gov:82/perl/snp/snp_cgi.pl) have also been discovered experimentally, see, Cox et al. Hum Mutal 2001, 17:141-150. However, these in silico methods could only find 27% of true SNPs.

The most common SNP typing methods currently include hybridization, primer extension and cleavage methods. Each of these methods must be connected to an appropriate detection system. Detection technologies include fluorescent polarization, (see Chan X et al. Genome Res 1999, 9:492-499), luminometric detection of pyrophosphate release (pyrosequencing), (see Ahmadiian A et al. Anal Biochem 2000, 280:103-10), fluorescence resonance energy transfer (FRET)-based cleavage assays, DHPLC, and mass spectrometry, (see Shi M M, Clin Chem 2001, 47:164-172 and U.S. Pat. No. 6,300,076 B1). Other methods of detecting and characterising SNPs are those disclosed in U.S. Pat. Nos. 6,297,018 B1 and 6,300,063 B1. The disclosures of the above references are incorporated herein by reference in their entirety.

In a particularly preferred embodiment the detection of the polymorphism can be accomplished by means of so called INVADER™ technology (available from Third Wave Technologies Inc. Madison, Wis.). In this assay, a specific upstream “invader” oligonucleotide and a partially overlapping downstream probe together form a specific structure when bound to complementary DNA template. This structure is recognized and cut at a specific site by the Cleavase enzyme, and this results in the release of the 5′ flap of the probe oligonucleotide. This fragment then serves as the “invader” oligonucleotide with respect to synthetic secondary targets and secondary fluorescently labeled signal probes contained in the reaction mixture. This results in specific cleavage of the secondary signal probes by the Cleavase enzyme. Fluoresence signal is generated when this secondary probe, labeled with dye molecules capable of fluorescence resonance energy transfer, is cleaved. Cleavases have stringent requirements relative to the structure formed by the overlapping DNA sequences or flaps and can, therefore, be used to specifically detect single base pair mismatches immediately upstream of the cleavage site on the downstream DNA strand. See Ryan D et al. Molecular Diagnosis Vol. 4 No 2 1999:135-144 and Lyamichev V et al. Nature Biotechnology Vol 17 1999:292-296, see also U.S. Pat. Nos. 5,846,717 and 6,001,567 (the disclosures of which are incorporated herein by reference in their entirety).

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

TCF1 genotyping oligonucleotides of the invention may also be immobilized on or synthesized on a solid surface such as a microchip, bead. or glass slide (see, e.g., WO 98/20020 and WO 98/20019). Such immobilized genotyping oligonucleotides may be used in a variety of polymorphism detection assays, including but not limited to probe hybridization and polymerase extension assays. Immobilized TCF1 genotyping oligonucleotides of the invention may comprise an ordered array of oligonucleotides designed to rapidly screen a DNA sample for polymorphisms in multiple genes at the same time.

An allele-specific oligonucleotide primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. Allele-specific oligonucleotide primers hybridizing to either the coding or noncoding strand are contemplated by the invention. An ASO primer for detecting TCF1 gene polymorphisms could be developed using techniques known to those of skill in the art.

Other genotyping oligonucleotides of the invention hybridize to a target region located one to several nucleotides downstream of one of the novel polymorphic sites identified herein. Such oligonucleotides are useful in polymerase-mediated primer extension methods for detecting one of the novel polymorphisms described herein and therefore such genotyping oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site.

In another embodiment, the invention provides a kit comprising at least two genotyping oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR.

The above described oligonucleotide compositions and kits are useful in methods for genotyping and/or haplotyping the TCF1 gene in an individual. As used herein, the terms “TCF1 genotype” and “TCF1 haplotype” mean the genotype or haplotype containing the nucleotide pair or nucleotide, respectively, that is present at one or more of the novel polymorphic sites described herein and may optionally also include the nucleotide pair or nucleotide present at one or more additional polymorphic sites in the TCF1 gene. The additional polymorphic sites may be currently known polymorphic sites or sites that are subsequently discovered.

One embodiment of the genotyping method involves isolating from the individual a nucleic acid mixture comprising the two copies of the TCF1 gene, or a fragment thereof, that are present in the individual, and determining the identity of the nucleotide pair at one or more of the polymorphic sites in the two copies to assign a TCF1 genotype to the individual. As will be readily understood by the skilled artisan, the two “copies” of a gene in an individual may be the same allele or may be different alleles. In a particularly preferred embodiment, the genotyping method comprises determining the identity of the nucleotide pair at each polymorphic site.

Typically, the nucleic acid mixture is isolated from a biological sample taken from the individual, such as a blood sample or tissue sample. Suitable tissue samples include whole blood, semen, saliva, tears, urine, fecal material, sweat, buccal smears, skin and hair. The nucleic acid mixture may be comprised of genomic DNA, mRNA, or cDNA and, in the latter two cases, the biological sample must be obtained from an organ in which the TCF1 gene is expressed. Furthermore it will be understood by the skilled artisan that mRNA or cDNA preparations would not be used to detect polymorphisms located in introns or in 5′ and 3′ nontranscribed regions. If a TCF1 gene fragment is isolated, it must contain the polymorphic site(s) to be genotyped.

One embodiment of the haplotyping method comprises isolating from the individual a nucleic acid molecule containing only one of the two copies of the TCF1 gene, or a fragment thereof, that is present in the individual and determining in that copy the identity of the nucleotide at one or more of the polymorphic sites in that copy to assign a TCF1 haplotype to the individual. The nucleic acid may be isolated using any method capable of separating the two copies of the TCF1 gene or fragment, including but not limited to, one of the methods described above for preparing TCF1 isogenes, with targeted in vivo cloning being the preferred approach. As will be readily appreciated by those skilled in the art, any individual clone will only provide haplotype information on one of the two TCF1 gene copies present in an individual. If haplotype information is desired for the individual's other copy, additional TCF1 clones will need to be examined. Typically, at least five clones should be examined to have more than a 90% probability of haplotyping both copies of the TCF1 gene in an individual. In a particularly preferred embodiment, the nucleotide at each of polymorphic site is identified.

In a preferred embodiment, a TCF1 haplotype pair is determined for an individual by identifying the phased sequence of nucleotides at one or more of the polymorphic sites in each copy of the TCF1 gene that is present in the individual. In a particularly preferred embodiment, the haplotyping method comprises identifying the phased sequence of nucleotides at each polymorphic site in each copy of the TCF1 gene. When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both the genotyping and haplotyping methods, the identity of a nucleotide (or nucleotide pair) at a polymorphic site(s) may be determined by amplifying a target region(s) containing the polymorphic site(s) directly from one or both copies of the TCF1 gene, or fragment thereof, and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a she may be positively determined to be either guanine or cytosine for ail individual homozygous at that site, or both guanine and cytosine, if the individual is heterozygous at that site. Alternatively, the site may be negatively determined to be not guanine (and thus cytosine/cytosine) or not cytosine (and thus guanine/guanine).

In addition, the identity of the allele(s) present at any of the novel polymorphic sites described herein may be indirectly determined by genotyping a polymorphic site not disclosed herein that is in linkage disequilibrium with the polymorphic site that is of interest. Two sites are said to be in linkage disequilibrium if the presence of a particular variant at one she enhances the predictability of another variant at the second site (See, Stevens, J C 1999, Mol Diag 4:309-317). Polymorphic sites in linkage disequilibrium with the presently disclosed polymorphic sites may be located in regions of the gene or in other genomic regions not examined herein. Genotyping of a polymorphic site in linkage disequilibrium with the novel polymorphic sites described herein may be performed by, but is not limited to, any of the above-mentioned methods for detecting the identity of the allele at a polymorphic site.

The target region(s) may be amplified using any oligonucleotide-directed amplification method, including but not limited to polymerase chain reaction (PCR) (U.S. Pat. No. 4,965,188), ligase chain reaction (LCR) (Barany et al., Proc Natl Acad Sci USA 88:189-193, 1991; WO 90/01069), and oligonucleotide ligation assay (OLA) (Landegren et al., Science 241:1077-1080, 1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, WO 89/06700) and isothermal methods (Walker et al., Proc Natl Acad Sci USA 89:392-396, 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C. and more preferably within 2° C., of each other when hybridizing to each of the polymorphic sites being detected.

Hybridization of an allele-specific oligonucleotide to a target polynucleotide may be performed with both entities in solution or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype or haplotype for the TCF1 gene of an individual may also be determined by hybridization of a nucleic sample containing one or both copies of the gene to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., Proc Natl Acad Sci USA 82:7575, 1985; Meyers et al., Science 230:1242, 1985) and proteins which recognize nucleotide mismatches, such as the E. coli mutS protein (Modrich P. Ann Rev Genet 25:229-253, 1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et at., Nucl Acids Res 18:2699-2706, 1990; Sheffield et al., Proc Natl Acad Sci USA 86:232-236, 1989).

A polymerase-mediated primer extension method may also be used to identify the polymorphism(s). Several such methods have been described in the patent and scientific literature and include the “Genetic Bit Analysis” method (WO 92/15712) and the ligase/polymerase mediated genetic bit analysis (U.S. Pat. No. 5,679,524). Related methods are disclosed in WO 91/02087, WO 90/09455, WO 95/17676, U.S. Pat. Nos. 5,302,509 and 5,945,283. Extended primers containing a polymorphism may be detected by mass spectrometry as described in U.S. Pat. No. 5,605,798. Another primer extension method is allele-specific PCR (Ruafio et al., Nucl Acids Res 17:8392, 1989; Ruafio et al., Nucl Acids Res 19, 6877-6882, 1991; WO 93/22456; Turki et al., I Clin Invest 95:1635-1641, 1995). In addition, multiple polymorphic sites may be investigated by simultaneously amplifying multiple regions of the nucleic acid using sets of allele-specific primers as described in Wallace et al. (WO 89/10414).

In a preferred embodiment, the haplotype frequency data for each ethnogeographic group is examined to determine whether it is consistent with Hardy-Weinberg equilibrium. Hardy-Weinberg equilibrium (D. L. Hartl et al., Principles of Population Genomics, Sinauer Associates (Sunderland, Mass.), 3rd Ed., 1997) postulates that the frequency of finding the haplotype pair H₁/H₂ is equal to P_{H-W} (H₁/H₂)=2p(H₁) p (H₂) if H₁≠H₂ and P_{H-W} (H₁/H₂)=p (H₁) p (H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology (U.S. Pat. No. 5,866,404), SMD, or allele-specific long-range PCR (Michalotos-Beloin et al., Nucl Acids Res 24:4841-4843, 1996).

In one embodiment of this method for predicting a TCF1 haplotype pair, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. In rare cases, either no haplotypes in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., Nucl Acids Res 24:4841-4843, 1996).

The invention also provides a method for determining the frequency of a TCF1 genotype or TCF1 haplotype in a population. The method comprises determining the genotype or the haplotype pair for the TCF1 gene that is present in each member of the population, wherein the genotype or haplotype comprises the nucleotide pair or nucleotide detected at one or more of the polymorphic sites in the TCF1 gene, including but not limited to 483 A>G; and calculating the frequency any particular genotype or haplotype is found in the population. The population may be a reference population, a family population, a same sex population, a population group, a trait population (e.g., a group of individuals exhibiting a trait of interest such as a medical condition or response to a therapeutic treatment).

In another aspect of the invention, frequency data for TCF1 genotypes and/or haplotypes found in a reference population are used in a method for identifying an association between a trait and a TCF1 genotype or a TCF1 haplotype. The trait may be any detectable phenotype, including but not limited to susceptibility to a disease or response to a treatment The method involves obtaining data on the frequency of the genotype(s) or haplotype(s) of interest in a reference population as well as in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above.

In another embodiment, the frequency data for the reference and/or trait populations is obtained by accessing previously determined frequency data, which may be in written or electronic form. For example, the frequency data may be present in a database that is accessible by a computer. Once the frequency data is obtained, the frequencies of the genotype(s) or haplotype(s) of interest in the reference and trait populations are compared. In a preferred embodiment, the frequencies of all genotypes and/or haplotypes observed in the populations are compared. If a particular genotype or haplotype for the TCF1 gene is more frequent in the trait population than in the reference population at a statistically significant amount, then the trait is predicted to be associated with that TCF1 genotype or haplotype.

In a preferred embodiment statistical analysis is performed by the use of standard ANOVA tests with a Bonferoni correction and/or a bootstrapping method that simulates the genotype phenotype correlation many times and calculates a significance value. When many polymorphisms are being analyzed a correction to factor may be performed to correct for a significant association that might be found by chance. For statistical methods for use in the methods of this invention, see: Statistical Methods in Biology, 3^{rd} edition, Bailey N T J, Cambridge Univ. Press (1997); introduction to Computational Biology, Waterman M S, CRC Press (2000) and Bioinformatics, Baxevanis A D and Ouellette B F F editors (2001) John Wiley & Sons, Inc.

In a preferred embodiment of the method, the trait of interest is a clinical response exhibited by a patient to some therapeutic treatment, for example, response to a drug targeting TCF1 or response to a therapeutic treatment for a medical condition.

In another embodiment of the invention, a detectable genotype or haplotype that is in linkage disequilibrium with the TCF1 genotype or haplotype of interest may be used as a surrogate marker. A genotype that is in linkage disequilibrium with a TCF1 genotype may be discovered by determining if a particular genotype or haplotype for the TCF1 gene is more frequent in the population that also demonstrates the potential surrogate marker genotype than in the reference population at a statistically significant amount, then the marker genotype is predicted to be associated with that TCF1 genotype or haplotype and then can be used as a surrogate marker in place of the TCF1 genotype.

As used herein, “medical condition” includes but is not limited to any condition or disease manifested as one or more physical and/or psychological symptoms for which treatment is desirable, and includes previously and newly identified diseases and other disorders.

As used herein, the term “clinical response” means any or all of the following: a quantitative measure of the response, no response, and adverse response (i.e., side effects).

In order to deduce a correlation between clinical response to a treatment and a TCF1 genotype or haplotype, it is necessary to obtain data on the clinical responses exhibited by a population of individuals who received the treatment, hereinafter the “clinical population”. This clinical data may be obtained by analyzing the results of a clinical trial that has already been run and/or the clinical data may be obtained by designing and carrying out one or more new clinical trials.

As used herein, the term “clinical trial” means any research study designed to collect clinical data on responses to a particular treatment, and includes but is not limited to phase I, phase II and phase III clinical trials. Standard methods are used to define the patient population and to enroll subjects.

It is preferred that the individuals included in the clinical population have been graded for the existence of the medical condition of interest. This is important in cases where the symptom(s) being presented by the patients can be caused by more than one underlying condition, and where treatment of the underlying conditions are not the same. An example of this would be where patients experience breathing difficulties that are due to either asthma or respiratory infections. If both sets were treated with an asthma medication, there would be a spurious group of apparent non-responders that did not actually have asthma. These people would affect the ability to detect any correlation between haplotype and treatment outcome. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the TCF1 gene for each individual in the trial population is genotyped and/or haplotyped, which may be done before or after administering the treatment.

After both the clinical and polymorphism data have been obtained, correlations between individual response and TCF1 genotype or haplotype content are created. Correlations may be produced in several ways. In one method, individuals are grouped by their TCF1 genotype or haplotype (or haplotype pair) (also referred to as a polymorphism group), and then the averages and standard deviations of clinical responses exhibited by the members of each polymorphism group are calculated.

These results are then analyzed to determine if any observed variation in clinical response between polymorphism groups is statistically significant. Statistical analysis methods which may be used are described in L. D. Fisher and G. vanBelle, “Biostatistics: A Methodology for the Health Sciences”, Wiley-Interscience (New York) 1993. This analysis may also include a regression calculation of which polymorphic sites in the TCF1 gene give the most significant contribution to the differences in phenotype. One regression model useful in the invention is described in the PCT Application entitled “Methods for Obtaining and Using Haplotype Data”, filed Jun. 26, 2000.

A second method for finding correlations between TCF1 haplotype content and clinical responses uses predictive models based on error-minimizing optimization algorithms. One of many possible optimization algorithms is a genetic algorithm (R. Judson, “Genetic Algorithms and Their Uses in Chemistry” in Reviews in Computational Chemistry, Vol. 10, pp. 1-73, K. B. Lipkowitz and D. B. Boyd, eds. (VCH Publishers, New York, 1997). Simulated annealing (Press et al., “Numerical Recipes in C: The Art of Scientific Computing”, Cambridge University Press (Cambridge) 1992, Ch. 10), neural networks (E. Rich and K. Knight, “Artificial intelligence”, 2nd Edition (McGraw-Hill, New York, 1991, Ch. 18), standard gradient descent methods (Press et al., supra Ch. 10), or other global or local optimization approaches (see discussion in Judson, supra) could also be used. Preferably, the correlation is found using a genetic algorithm approach as described in PCT Application entitled “Methods for Obtaining and Using Haplotype Data”, filed Jun. 26, 2000.

Correlations may also be analyzed using analysis of variation (ANOVA) techniques to determine how much of the variation in the clinical data is explained by different subsets of the polymorphic sites in the TCF1 gene. As described in PCT Application entitled “Methods for Obtaining and Using Haplotype Data”, filed Jun. 26, 2000, ANOVA is used to test hypotheses about whether a response variable is caused by or correlated with one or more traits or variables that can be measured (Fisher and vanBelle, supra, Ch. 10).

From the analyses described above, a mathematical model may be readily constructed by the skilled artisan that predicts clinical response as a function of TCF1 genotype or haplotype content. Preferably, the model is validated in one or more follow-up clinical trials designed to test the model.

The identification of an association between a clinical response and a genotype or haplotype (or haplotype pair) for the TCF1 gene may be the basis for designing a diagnostic method to determine those individuals who will or will not respond to the treatment, or alternatively, will respond at a lower level and thus may require more treatment, i.e., a greater dose of a drug. The diagnostic method may take one of several forms: for example, a direct DNA test (i.e., genotyping or haplotyping one or more of the polymorphic sites in the TCF1 gene), a serological test, or a physical exam measurement. The only requirement is that there be a good correlation between the diagnostic test results and the underlying TCF1 genotype or haplotype that is in turn correlated with the clinical response. In a preferred embodiment, this diagnostic method uses the predictive haplotyping method described above.

A computer may implement any or all analytical and mathematical operations involved in practicing the methods of the present invention. In addition, the computer may execute a program that generates views (or screens) displayed on a display device and with which the user can interact to view and analyze large amounts of information relating to the TCF1 gene and its genomic variation, including chromosome location, gene structure, and gene family, gene expression data, polymorphism data, genetic sequence data, and clinical data population data (e.g., data on ethnogeographic origin, clinical responses, genotypes, and haplotypes for one or more populations). The TCF1 polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

In other embodiments, the invention provides methods, compositions, and kits for haplotyping and/or genotyping the TCF1 gene in an individual. The methods involve identifying the nucleotide or nucleotide pair present at nucleotide: 483 A>G in from GenBank accession number U72616. This nucleotide substitution changes the amino acid Asn 487 Ser in one or both copies of the TCF1 gene from the individual. The compositions contain oligonucleotide probes and primers designed to specifically hybridize to one or more target regions containing, or that are adjacent to, a polymorphic site. The methods and compositions for establishing the genotype or haplotype of an individual at the novel polymorphic sites described herein are useful for studying the effect of the polymorphisms in the etiology of diseases affected by the expression and function of the TCF1 protein, studying the efficacy of drugs targeting TCF1, predicting individual susceptibility to diseases affected by the expression and function of the TCF1 protein and predicting individual responsiveness to drugs targeting TCF1.

In yet another embodiment, the invention provides a method for identifying an association between a genotype or haplotype and a trait in preferred embodiments, the trait is susceptibility to a disease, severity of a disease, the staging of a disease or response to a drug. Such methods have applicability in developing diagnostic tests and therapeutic treatments for all pharmacogenetic applications where there is the potential for an association between a genotype and a treatment outcome including efficacy measurements, PK measurements and side effect measurements.

The present invention also provides a computer system for storing and displaying polymorphism data determined for the TCF1 gene. The computer system comprises a computer processing unit; a display; and a database containing the polymorphism data. The polymorphism data includes the polymorphisms, the genotypes and the haplotypes identified for the TCF1 gene in a reference population. In a preferred embodiment, the computer system is capable of producing a display showing TCF1 haplotypes organized according to their evolutionary relationships.

In another aspect, the invention provides SNP probes, which are useful in classifying people according to their types of genetic variation. The SNP probes according to the invention are oligonucleotides, which can discriminate between alleles of a SNP nucleic acid in conventional allelic discrimination assays.

As used herein, a “SNP nucleic acid” is a nucleic acid sequence, which comprises a nucleotide that is variable within an otherwise identical nucleotide sequence between individuals or groups of individuals, thus, existing as alleles. Such SNP nucleic acids are preferably from about 15 to about 500 nucleotides in length. The SNP nucleic acids may be part of a chromosome, or they may be an exact copy of a part of a chromosome, e.g., by amplification of such a part of a chromosome through PCR or through cloning. The SNP nucleic adds are referred to hereafter simply as “SNPs”. The SNP probes according to the invention are oligonucleotides that are complementary to a SNP nucleic acid.

As used herein, the term “complementary” means exactly complementary throughout the length of the oligonucleotide in the Watson and Crick sense of the word.

In certain preferred embodiments, the oligonucleotides according to this aspect of the invention are complementary to one allele of the SNP nucleic acid, but not to any other allele of the SNP nucleic acid. Oligonucleotides according to this embodiment of the invention can discriminate between alleles of the SNP nucleic acid in various ways. For example, under stringent hybridization conditions, an oligonucleotide of appropriate length will hybridize to one allele of the SNP nucleic add, but not to any other allele of the SNP nucleic acid. The oligonucleotide may be labeled by a radiolabel or a fluorescent label. Alternatively, an oligonucleotide of appropriate length can be used as a primer for PCR, wherein the 3′ terminal nucleotide is complementary to one allele of the SNP nucleic acid, but not to any other allele. In this embodiment, the presence or absence of amplification by PCR determines the haplotype of the SNP nucleic acid

Thus, in one embodiment, the invention provides an isolated polynucleotide comprising a nucleotide sequence that is a polymorphic variant of a reference sequence for the TCF1 gene or a fragment thereof. The reference sequence comprises UniGene Cluster Hs.73888 and the polymorphic variant comprises at least one polymorphism, including but not limited to nucleotide: 483 A>G. A particularly preferred polymorphic variant is a naturally-occurring isoform (also referred to herein as an “isogene”) of the TCF1 gene.

Genomic and cDNA fragments of the invention comprise at least one novel polymorphic site identified herein and have a length of at least 10 nucleotides and may range up to the full length of the gene. Preferably, a fragment according to the present invention is between 100 and 3000 nucleotides in length, and more preferably between 200 and 2000 nucleotides in length, and most preferably between 500 and 1000 nucleotides in length

In describing the polymorphic sites identified herein reference is made to the sense strand of the gene for convenience. However, as recognized by the skilled artisan, nucleic acid molecules containing the TCF1 gene may be complementary double stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the TCF1 genomic variants described herein.

In a further aspect of the invention there is provided a kit for the identification of a patient's polymorphism pattern at the TCF1 polymorphic site at 483 A>G, said kit comprising a means for determining a genetic polymorphism pattern at the TCF1 polymorphic site at 483 A>G.

In a preferred embodiment, such kit may further comprise a DNA sample collecting means.

In a preferred embodiment the means for determining a genetic polymorphism pattern at the TCF1 polymorphic site at 483 A>G comprise at least one TCF1 genotyping oligonucleotide. In particular, the means for determining a genetic polymorphism pattern at the TCF1 polymorphic site at 483 A>G may comprise two TCF1 genotyping oligonucleotides. Also, the means for determining a genetic polymorphism pattern at the TCF1 polymorphic site at 483 A>G may comprise at least one TCF1 genotyping primer compositon comprising at least one TCF1 genotyping oligonucleotide. In particular, the TCF1 genotyping primer compositon may comprise at least two sets of allele specific primer pairs. Preferably, the two TCF1 genotyping oligonucleotides are packaged in separate containers.

It is to be understood that the methods of the invention described herein generally may further comprise the use of a kit according to the invention. Generally, the methods of the invention may be performed ex-vivo, and such ex-vivo methods are specifically contemplated by the present invention. Also, where a method of the invention may include steps that may be practised on the human or animal body, methods that only comprise those steps which are not practised on the human or animal body are specifically contemplated by the present invention.

Effect(s) of the polymorphisms identified herein on expression of TCF1 may be investigated by preparing recombinant cells and/or organisms, preferably recombinant animals, containing a polymorphic variant of the TCF1 gene. As used herein, “expression” includes but is not limited to one or more of the following: transcription of the gene into precursor mRNA; splicing and other processing of the precursor mRNA to produce mature mRNA; mRNA stability; translation of the mature mRNA into TCF1 protein (including codon usage and tRNA availability); and glycosylation and/or other modifications of the translation product, if required for proper expression and function.

To prepare a recombinant cell of the invention, the desired TCF1 isogene may be introduced into the cell in a vector such that the isogene remains extrachromosomal. In such a situation, the gene will be expressed by the cell from the extrachromosomal location. In a preferred embodiment, the TCF1 isogene is introduced into a cell in such a way that it recombines with the endogenous TCF1 gene present in the cell. Such recombination requires the occurrence of a double recombination event, thereby resulting in the desired TCF1 gene polymorphism. Vectors for the introduction of genes both for recombination and for extrachromosomal maintenance are known in the art, and any suitable vector or vector construct may be used in the invention. Methods such as electroporation, particle bombardment, Calcium phosphate co-precipitation and viral transduction for introducing DNA into cells are known in the art; therefore, the choice of method may lie with the competence and preference of the skilled practitioner.

Examples of cells into which the TCF1 isogene may be introduced include, but are not limited to, continuous culture cells, such as COS, NIH/3T3, and primary or culture cells of the relevant tissue type, i.e., they express the TCF1 isogene. Such recombinant cells can be used to compare the biological activities of the different protein variants.

Recombinant organisms, i.e., transgenic animals, expressing a variant TCF1 gene are prepared using standard procedures known in the art. Preferably, a construct comprising the variant gene is introduced into a nonhuman animal or an ancestor of the animal at an embryonic stage, i.e., the one-cell stage, or generally not later than about the eight-cell stage. Transgenic animals carrying the constructs of the invention can be made by several methods known to those having skill in the art. One method involves transfecting into the embryo a retrovirus constructed to contain one or more insulator elements, a gene or genes of interest, and other components known to those skilled in the art to provide a complete shuttle vector harboring the insulated gene(s) as a transgene, see e.g., U.S. Pat. No. 5,610,053. Another method involves directly injecting a transgene into the embryo. A third method involves the use of embryonic stem cells.

Examples of animals, into which the TCF1 isogenes may be introduced include, but are not limited to, mice, rats, other rodents, and nonhuman primates (see “The introduction of Foreign Genes into Mice” and the cited references therein, in: Recombinant DNA, Eds. J. D. Watson, M. Gilman, J. Witkowski, and M. Zoller; W. H. Freeman and Company, New York, pages 254-272). Transgenic animals stably expressing a human TCF1 isogene and producing human TCF1 protein can be used as biological models for studying diseases related to abnormal TCF1 expression and/or activity, and for screening and assaying various candidate drugs, compounds, and treatment regimens to reduce the symptoms or effects of these diseases.

In addition, treatment with a glycemic control agent or therapy can be used in subjects with impaired glycemic control, including: type 2 and type 1 diabetes, impaired glucose metabolism (impaired glucose tolerance and/or impaired fasting glucose), Syndrome X, prandial lipemia, gestational diabetes, for the prevention or delay of progression to overt diabetes mellitus type 2; for the prevention, reduction or delay in onset of a condition selected from the group consisting of increased microvascular complications; increased cardiovascular morbidity; excess cerebrovascular diseases; increased cardiovascular mortality and sudden death; higher incidences and mortality rates of malignant neoplasms; and other metabolic disturbances that are associated with IGM.

Furthermore, glycemic control agents or therapies can be used in subjects with impaired glycemic control (IGC) for the prevention, reduction or delay in onset of a condition selected from the group e.g. consisting of retinopathy, other ophthalmic complications of diabetes, nephropathy, neuropathy, peripheral angiopathy, peripheral angiopathy, gangrene, myocardial infarctions, coronary heart disease, atherosclerosis, other acute and subacute forms of coronary ischemia, stroke, dyslipidemia, hyperuricemia, hypertension, angina pectoris, microangiopathic changes that result in amputation, cancer, cancer deaths, obesity, uricemia, insulin resistance, arterial occlusive disease, and atherosclerosis.

According to the present invention, glycemic control agents or therapies agents can be used in subjects with IGC, to prevent or delay the progression to overt diabetes, to reduce microvascular complications of diabetes, to reduce vascular, especially cardiovascular, mortality and morbidity, especially cardiovascular morbidity and mortality, and to reduce increased mortality related to cancer in individuals with IGC.

Accordingly, the present invention relates to a method in subjects with IGC, for the prevention or delay of progression to overt diabetes mellitus type 2; for the prevention, reduction or delay in onset of a condition selected from the group consisting of increased microvascular complications; increased cardiovascular morbidity; excess cerebrovascular diseases; increased cardiovascular mortality and sudden death; higher incidences and mortality rates of malignant neoplasms; and other metabolic disturbances that are associated with IGC. Especially, the present invention relates to a method used in subjects with IGC, for the prevention, reduction or delay in onset of a condition selected from the group e.g. consisting of retinopathy, other ophthalmic complications of diabetes, nephropathy, neuropathy, peripheral angiopathy, peripheral angiopathy gangrene, myocardial infarctions, coronary heart disease, atherosclerosis, other acute and subacute forms of coronary ischemia, stroke, dyslipidemia, hyperuricemia, hypertension, angina pectoris, microangiopathic changes that result in amputation, cancer, cancer deaths, obesity, uricemia, insulin resistance, arterial occlusive disease, and atherosclerosis.

Accordingly, the present invention relates to a method of prevention or delay of the progression to overt diabetes, especially type 2 (ICD-9 Code 250.2), prevention or reduction of microvascular complications like retinopathy (ICD-9 code 250.5), neurophathy (ICD-9 code 250.6), nephropathy (ICD-9 code 250.4) and peripheral angiopathy or gangrene (ICD-9 code 250.7), later termed “microvascular complications” in subjects with IGM, especially IFG and IGT. Further the present invention relates to a method to prevent or reduce conditions of excessive cardiovascular morbidity (ICD-9 codes 410-414), e.g. myocardial infarction (ICD-9 code 410), arterial occlusive disease, atherosclerosis and other acute and subacute forms of coronary ischemia (ICD-9 code 411-414), later termed “cardiovascular morbidity”; to prevent, reduce, or delay the onset of excess cerebrovascular diseases like stroke (ICD-9 codes 430-438); to reduce increased cardiovascular mortality (ICD-9 codes 390-459) and sudden death (ICD-9 code 798.1); to prevent the development of cancer (ICD-9 codes 140-208) and to reduce cancer deaths, in each case, in subjects with IGC.

The method further relates to a method of prevention or reduction of other metabolic disturbances that are associated with IGC including hyperglycemia (including isolated postprandial hyperglycemia), dyslipidemia (ICD-9 code 272), hyperuricemia (ICD-9 code 790.6) as well as hypertension (ICD-9 codes 401-404) and angina pectoris (ICD-9 code 413.9), in each case, in subjects with IGC. The codes identified hereinbefore and herafter according to the international Classification of Diseases 9th version and the corresponding definitions allocated thereto are herewith incorporated by reference and likewise form part of the present invention.

The method comprises administering to a subject in need thereof an effective amount of a glycemic control agents or therapies or a pharmaceutically acceptable salt of such an agent or compound. A subject in need of such method is a warm-blooded animal including man. The present invention also relates to a method to be used in subjects with IGC, and associated diseases and conditions such as isolated prandial hyperglycemia, prevention or delay of the progression to overt diabetes, especially type 2, prevention, reduction, or delay the onset of microvascular complications, prevention or reduction of gangrene or microangiopathic changes that result in amputation, prevention or reduction of excessive cardiovascular morbidity and cardiovascular mortality, prevention of cancer and reduction of cancer deaths.

The present invention likewise relates to a method of treatment of conditions and diseases associated with IGC (including isolated prandial hyperglycemia) including obesity, increased age, diabetes during pregnancy, dyslipidemia, high blood pressure, uricemia, insulin resistance, arterial occlusive disease, atherosclerosis, retinopathy, nephropathy, angina pectoris, myocardial infarction, and stroke. Preferably, said preventions should be effected in individuals with glucose levels in the ranges that have been proven in large epidemiologic studies to confer increased cardiovascular, microvascular and cancer risk. These levels include levels of plasma glucose 7.8 mmol/L mmol/L after an OGTT or casual glucose evaluation and/or fasting plasma glucose in the IFG range (fasting plasma glucose between 6.1 and 7 mmol/L). As new epidemiologic data become available to lower the glycemic levels that are incontrovertibly linked to the above-mentioned risks, or as the international standards for defining the risk groups are changed, the use of the invention is also warranted for treatment of the groups at risk.

The present invention also relates to a method to be used in subjects with IFG comprising administering to a subject in need thereof a therapeutically effective amount of a glycemic control agents, including but not limited to a DPP-IV inhibitor.

The present invention relates to the use of a glycemic control agents or a pharmaceutically acceptable salt thereof for the manufacture of a medicament in subjects with IGC, for the prevention or delay of progression to overt diabetes mellitus type 2; for the prevention, reduction or delay in onset of a condition selected from the group consisting of increased microvascular complications; increased cardiovascular morbidity; excess cerebrovascular diseases; increased cardiovascular mortality and sudden death; higher incidences and mortality rates of malignant neoplasms; and other metabolic disturbances that are associated with IGC.

The present invention relates to the use of an glycemic control agent including a DPP4 inhibitor or a pharmaceutically acceptable salt for the manufacture of a medicament in subjects with IGC, and associated diseases and conditions such as isolated prandial hyperglycemia for the following: prevention or delay of the progression to overt diabetes, especially type 2, prevention or reduction of microvascular complications, prevention or reduction of excessive cardiovascular morbidity and cardiovascular mortality, prevention of cancer and reduction of cancer deaths.

The corresponding active ingredient or a pharmaceutically acceptable salt thereof may also be used in form of a hydrate or include other solvents used for crystallization. Furthermore, the present invention relates to the combination such as a combined preparation or pharmaceutical composition, respectively, comprising more than one glycemic control agents to be used in subjects with IGM, especially IFG and/or IGT, for the prevention or delay of progression to overt diabetes mellitus type 2; for the prevention, reduction or delay in onset of a condition selected from the group consisting of increased microvascular complications; increased cardiovascular morbidity; excess cerebrovascular diseases; increased cardiovascular mortality and sudden death; higher incidences and mortality rates of malignant neoplasms; and other metabolic disturbances that are associated with IGM.

Further benefits when applying the combination of the present invention are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated. Preferably, the jointly therapeutically effective amounts of the active agents according to the combination of the present invention can be administered simultaneously or sequentially in any order, separately or in a fixed combination.

The term “therapeutically effective amount” as used herein, shall mean that amount of a drug or combination that will elicit the biological or medical response needed to achieve the therapeutic effect as specified according to the present invention in the warm-blooded animal, including man. A “therapeutically effective amount” can be administered when administering a single agent and also in both a fixed or free combination of two or more compounds.

A “jointly effective amount” as used herein, shall mean an amount of one or more components of a combination that may be non-effective by itself but when used in a combination according to the present invention may be therapeutically effective in combination with one or more other agents if the overall therapeutic effect can be achieved by the combined administration of the (fixed or free) multiple agents. The pharmaceutical composition according to the present invention as described hereinbefore and hereinafter may be used for simultaneous use or sequential use in any order, for separate use or as a fixed combination.

Preferred glycemic control agents include, but are not limited to, DPP4 inhibitors such as the compounds; 2-Pyrrolidinecarbonitrile, 1-[[[2-[(5-cyano-2-pyridinyl) amino]ethyl]amino]acetyl]-, (2S) and (1-[3Hydroxy-adamant-1-ylamino)-acetyl]-pyrrolidine-2(S)-carbonitrile) or, if appropriate, in each case, a pharmaceutically acceptable salt thereof.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components. The invention furthermore relates to a commercial package comprising the combination according to the present invention together with instructions for simultaneous, separate or sequential use. The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases. Pharmaceutically acceptable salts are for example, salts formed with bases, namely cationic salts such as alkali and alkaline earth metal salts, as well as ammonium salts.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carries, especially suitable for enteral or parenteral application.

The novel pharmaceutical preparations contain, for example, from about 10% to about 100%, preferably 80%, most preferably from about 90% to about 99%, of the active ingredient. Pharmaceutical preparations according to the invention for enteral or parenteral administration are, for example, those in unit dose forms, such as sugar-coated tablets, tablets, capsules or suppositories, or ampoules. These are prepared in a manner well known to one of skill in the art, for example by means of conventional mixing, granulating, sugarcoating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

The precise dosage of the compounds of the present invention, and their corresponding pharmaceutically acceptable acid addition salts, to be employed for treating conditions or disorders characterized by impaired glycemic control depends upon several factors, including the host, the nature and the severity of the condition being treated, the mode of administration and the particular compound employed. However, in general, conditions or disorders characterized by impaired glycemic control are effectively treated when a compound of the invention, or a corresponding pharmaceutically acceptable acid addition salt, is administered enterally, e.g., orally, or parenterally, e.g., intravenously, but preferably orally, at a daily dosage of 0.002-10 mg/kg body weight, preferably 0.02-2.5 mg/kg body weight or, for most larger primates, a daily dosage of 0.1-250, preferably 1-100 mg. A typical oral dosage unit is 0.01-0.75 mg/kg, one to three times a day.

Usually, a small dose is administered initially and the dosage is gradually increased until the optimal dosage for the host under treatment is determined. The upper limit of dosage is that imposed by side effects and can be determined by trial for the host being treated.

The compounds of the present invention, and their corresponding pharmaceutically acceptable acid addition salts, may be combined with one or more pharmaceutically acceptable carriers and, optionally, one or more other conventional pharmaceutical adjuvants and administered enterally, e.g., orally, in the form of tablets, capsules, caplets, etc. or parenterally, e.g., intravenously, in the form of sterile injectable solutions or suspensions. The enteral and parenteral compositions may be prepared by conventional means.

The compounds of the present invention, and their corresponding pharmaceutically acceptable acid addition salts, may be formulated into enteral and parenteral pharmaceutical compositions containing an amount of the active substance that is effective for treating conditions or disorders characterized by impaired glycemic control and a pharmaceutically acceptable carrier such compositions may be formulated in unit dosage form.

The compounds of the present invention (including those of each of the subscopes thereof and each of the examples) may be administered in enantiomerically pure form (e.g., purity greater that 98% and preferably greater than 99% of one enantiomer) or with both enantiomers present together, e.g., in racemic form. The above dosage ranges are based on a single enantiomer of the compounds of the present invention. (excluding the amount of the less active enantiomer, if any).

A person skilled in the art is fully enabled, based on his knowledge, to determine the specific doses for the specific glycemic control agent, including DPP4 inhibitors, whether taken alone or in combination.

### EXAMPLES

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### Example 1

A 40 year old woman is found, on routine screening, to have an elevated blood glucose level. Her physician performs an oral glucose tolerance test and determines that the patient has impaired glucose tolerance. The physician discusses with the patient the short- and long-term consequences of impaired glucose tolerance and the possibility of progression to overt diabetes. The physician also discusses the available treatment modalities including diet, weight loss, exercise and medications including various glycemic control agents such as the DPP4 inhibitors then available. In addition, the physician counsels the patient about the possibility of testing her for the presence of the polymorphism in the TCF1 gene and explains what this result would mean with regard to the use of medication, including DPP4 inhibitors.

The patient agrees to the testing and the genotyping shows the presence of the GG genotype. On the basis of these results, the physician recommends and the patient agrees to a trial of a medication such as a DPP4 inhibitor to help correct her abnormal glucose tolerance and post-prandial hyperglycemia.

### Example 2

A 52 year old man with type II diabetes is seen by his physician. The patient is taking a glycemic control agent and glucose levels are in good control but the patient is experiencing numerous side effects from the medication. The physician recommends genotyping and counsels the patient regarding the treatment options that the genotyping results would allow. The patient is tested and determined to have the genotype associated with the most favorable response to DPP4 inhibitors. On the basis of this result and the expected high sensitivity to DDP4 inhibitors the physician is able to recommend a treatment regimen with a low dose of a DPP4 inhibitor with reduced likelihood of side effects. This treatment can supplement continued treatment with a reduced dose of the glycemic control agent this patient was previously treated with and was not able to tolerate or a low dose regimen of the DPP4 inhibitor alone can be substituted.

Definitions

As used herein, in the context of this disclosure, the following terms shall be defined as follows unless otherwise indicated:

Allele—A particular form of a genetic locus, distinguished from other forms by its particular nucleotide sequence.

Candidate gene—A gene which is hypothesized to be responsible for a disease, condition, or the response to a treatment, or to be correlated with one of these.

Gene—A segment of DNA that contains all the information for the regulated biosynthesis of an RNA product, including promoters, exons, introns, and other untranslated regions that control expression.

Genotype—An unphased 5′ to 3′ sequence of nucleotide pair(s) found at one or more polymorphic sites in a locus on a pair of homologous chromosomes in an individual. As used herein, genotype includes a full-genotype and/or a sub-genotype as described below.

Full-genotype—The unphased 5′ to 3′ sequence of nucleotide pairs found at all known polymorphic sites in a locus on a pair of homologous chromosomes in a single individual.

Sub-genotype—The unphased 5′ to 3′ sequence of nucleotides seen at a subset of the known polymorphic sites in a locus on a pair of homologous chromosomes in a single individual.

Genotyping—A process for determining a genotype of an individual.

Haplotype—A 5′ to 3′ sequence of nucleotides found at one or more polymorphic sites in a locus on a single chromosome from a single individual. As used herein, haplotype includes a full-haplotype and/or a sub-haplotype as described below.

Full-haplotype—The 5′ to 3′ sequence of nucleotides found at all known polymorphic sites in a locus on a single chromosome from a single individual.

Sub-haplotype—The 5′ to 3′ sequence of nucleotides seen at a subset of the known polymorphic sites in a locus on a single chromosome from a single individual.

Haplotype pair—The two haplotypes found for a locus in a single individual.

Haplotyping—A process for determining one or more haplotypes in an individual and includes use of family pedigrees, molecular techniques and/or statistical inference.

Haplotype data—information concerning one or more of the following for a specific gene: a listing of the haplotype pairs in each individual in a population; a listing of the different haplotypes in a population; frequency of each haplotype in that or other populations, and any known associations between one or more haplotypes and a trait.

Isoform—A particular form of a gene, mRNA, cDNA or the protein encoded thereby, distinguished from other forms by its particular sequence and/or structure.

Isogene—One of the isoforms of a gene found in a population. An isogene contains all of the polymorphisms present in the particular isoform of the gene.

Isolated—As applied to a biological molecule such as RNA, DNA, oligonucleotide, or protein, isolated means the molecule is substantially free of other biological molecules such as nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.

Linkage—describes the tendency of genes to be inherited together as a result of their location on the same chromosome; measured by percent recombination between loci.

Linkage disequilibrium—describes a situation in which some combinations of genetic markers occur more or less frequently in the population than would be expected from their distance apart. It implies that a group of markers has been inherited coordinately. It can result from reduced recombination in the region or from a founder effect, in which there has been insufficient time to reach equilibrium since one of the markers was introduced into the population.

Locus—A location on a chromosome or DNA molecule corresponding to a gene or a physical or phenotypic feature.

Naturally-occurring—A term used to designate that the object it is applied to, e.g., naturally-occurring polynucleotide or polypeptide, can be isolated from a source in nature and which has not been intentionally modified by man.

Nucleotide pair—The nucleotides found at a polymorphic site on the two copies of a chromosome from an individual.

Phased—As applied to a sequence of nucleotide pairs for two or more polymorphic sites in a locus, phased means the combination of nucleotides present at those polymorphic sites on a single copy of the locus is known.

Polymorphic site (PS)—A position within a locus at which at least two alternative sequences are found in a population, the most frequent of which has a frequency of no more than 99%.

Polymorphic variant—A gene, mRNA, cDNA, polypeptide or peptide whose nucleotide or amino acid sequence varies from a reference sequence due to the presence of a polymorphism in the gene.

Polymorphism—The sequence variation observed in an individual at a polymorphic site. Polymorphisms include nucleotide substitutions, insertions, deletions and microsatellites and may, but need not, result in detectable differences in gene expression or protein function.

Polymorphism data—information concerning one or more of the following for a specific gene: location of polymorphic sites; sequence variation at those sites; frequency of polymorphisms in one or more populations; the different genotypes and/or haplotypes determined for the gene; frequency of one or more of these genotypes and/or haplotypes in one or more populations; any known association(s) between a trait and a genotype or a haplotype for the gene.

Polymorphism database—A collection of polymorphism data arranged in a systematic or methodical way and capable of being individually accessed by electronic or other means.

Polynucleotide—A nucleic acid molecule comprised of single-stranded RNA or DNA or comprised of complementary, double-stranded DNA.

Population group—A group of individuals sharing a common characteristic such as ethnogeographic origin, medical condition, response to treatment etc.

Reference population—A group of subjects or individuals who are predicted to be representative of 1 or more characteristics of the population group. Typically, the reference population represents the genetic variation in the population at a certainty level of at least 85%, preferably at least 90%, more preferably at least 95% and even more preferably at least 99%.

Single Nucleotide Polymorphism (SNP)—Typically, the specific pair of nucleotides observed at a single polymorphic site. In rare cases, three or four nucleotides may be found.

Subject—A human individual whose genotypes or haplotypes or response to treatment or disease state are to be determined.

Treatment—A stimulus administered internally or externally to a subject.

Unphased—As applied to a sequence of nucleotide pairs for two or more polymorphic sites in a locus, unphased means the combination of nucleotides present at those polymorphic sites on a single copy of the locus is not known.

DPP4 inhibitor—as used herein, the term DPP4 inhibitor means a compound capable of inhibiting the catalytic actions of the enzyme DPP4 (DPP-IV; dipeptidylpeptidase IV; EC 3.4.14.5), which is a serine exopeptidase identical to ADA complexing protein-2 and to the T-cell activation antigen CD26.

References Cited

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarise the assertions made by their authors and no admission is made that any reference constitutes prior art. Applicants reserve the right to challenge the accuracy and pertinence of the cited references.

In addition, all GenBank accession numbers, Unigene Cluster numbers and protein accession numbers cited herein are incorporated herein by reference in their entirety and for all purposes to the same extent as if each such number was specifically and individually indicated to be incorporated by reference in its entirety for all purposes

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1. A method for determining the responsiveness of an individual with a disorder characterized by impaired glycemic control to treatment with a glycemic control agent or therapy, comprising;
(a) determining for the two copies of the TCF1 gene present in the individual, the identity of the nucleotide pair at the polymorphic site at 483 A>G, and
(b) assigning the individual to a good responder group if both pairs are GC or if one pair is AT and one pair is GC and to a low responder group if both pairs are AT.

2. The method of claim 1 wherein the glycemic control agent or therapy comprises administration of a dipeptidylpeptidase 4 (DPP4) inhibitor.

3. The method of claim 1 wherein the glycemic control agent or therapy comprises administration of 2-Pyrrolidinecarbonitrile, 1-[[[2-[(5-cyano-2-pyridinyl) amino]ethyl]amino]acetyl]-, (2S).

4. The method of claim 1 wherein the glycemic control agent or therapy comprises administration of 1-[3Hydroxy-adamant-1-ylamino)-acetyl]-pyrrolidine-2(S)-carbonitrile.

5. The method of claim 1 wherein the glycemic control agent or therapy is selected from the compounds of Formula I or Formula II.

6. The method of claim 1 wherein the disorder characterized by impaired glycemic control is type 2 diabetes mellitus.

7. The method of claim 1 wherein the disorder characterized by impaired glycemic control is type 1 diabetes mellitus.

8. The method of claim 1 wherein the disorder characterized by impaired glycemic control is impaired glucose tolerance.

9. The method of claim 1 wherein the disorder characterized by impaired glycemic control is impaired fasting glucose.

10. The method of claim 1 wherein the disorder characterized by impaired glycemic control is Syndrome X.

11. The method of claim 1 wherein the disorder characterized by impaired glycemic control is gestational diabetes.

12. The method of claim 1 wherein the disorder characterized by impaired glycemic control is impaired glucose metabolism (IGM).

13. The method of claim 1 wherein the disorder characterized by impaired glycemic control is a disorder responsive to DPP4 inhibitors

14. A method for treating an individual with a disorder characterized by impaired glycemic control, comprising,
(a) determining, for the two copies of the TCF1 gene present in the individual, the identity of the nucleotide pair at the polymorphic site 483 A>G, wherein,
(b) if both the nucleotide pairs are CG or if one is AT and one is CG the individual is treated with a glycemic control agent or therapy and if the nucleotide pairs are both AT the individual is treated with alternate therapy.

15. The method of claim 14 wherein the glycemic control agent or therapy comprises administration of a dipeptidylpeptidase 4 (DPP4) inhibitor.

16. The method of claim 14 wherein the glycemic control agent or therapy comprises administration of 2-Pyrrolidinecarbonitrile, 1-[[[2-[(5-cyano-2-pyridinyl) amino]ethyl]amino]acetyl]-, (2S).

17. The method of claim 14 wherein the glycemic control agent or therapy comprises administration of 1-[3Hydroxy-adamant-1-ylamino)-acetyl]-pyrrolidine-2(S)-carbonitrile.

18. The method of claim 14 wherein the glycemic control agent is selected from the compounds of Formula I or Formula II.

19. The method of claim 14 wherein the disorder characterized by impaired glycemic control is type 2 diabetes mellitus.

20. The method of claim 14 wherein the disorder characterized by impaired glycemic control is type 1 diabetes mellitus.

21. The method of claim 14 wherein the disorder characterized by impaired glycemic control is impaired glucose tolerance.

22. The method of claim 14 wherein the disorder characterized by impaired glycemic control is impaired fasting glucose.

23. The method of claim 14 wherein the disorder characterized by impaired glycemic control is Syndrome X.

24. The method of claim 14 wherein the disorder characterized by impaired glycemic control is gestational diabetes.

25. The method of claim 14 wherein the disorder characterized by impaired glycemic control is impaired glucose metabolism (IGM).

26. The method of claim 14 wherein the disorder characterized by impaired glycemic control is a disorder responsive to DPP4 inhibitors.

27. A method for identifying an association between a trait and at least one genotype or haplotype of the TCF1 gene which comprises, comparing the frequency of the genotype or haplotype in a population exhibiting the trait with the frequency of the genotype or haplotype in a reference population, wherein the genotype or haplotype comprises a nucleotide pair or nucleotide located at the polymorphic site 483 A>G, wherein a higher frequency of the genotype or haplotype in the trait population than in the reference population indicates the trait is associated with the genotype or haplotype.

28. The method of claim 26, wherein the trait is a clinical response to a drug targeting TCF1 or DPP4.

29. A method for treating an individual, with a disorder characterized by impaired glycemic control, comprising,
(a) determining, for the two copies of the TCF1 gene present in the individual, the identity of the nucleotide pair at the polymorphic site 483 A>G, wherein,
(b) if both the nucleotide pairs are CG or if one is AT and one is CG the individual is treated with a low dose of a glycemic control agent and if the nucleotide pairs are both AT the individual is treated with a high dose of a glycemic control agent.

30. The method of claim 29 wherein the glycemic control agent is a dipeptidylpeptidase 4 (DPP4) inhibitor.

31. The method of claim 29 wherein the glycemic control agent or therapy comprises administration of 2-Pyrrolidinecarbonitrile, 1-[[[2-[(5-cyano-2-pyridinyl) amino]ethyl]amino]acetyl]-, (2S).

32. The method of claim 29 wherein the glycemic control agent or therapy comprises administration of 1-[3Hydroxy-adamant-1-ylamino)-acetyl]-pyrrolidine-2(S)-carbonitrile.

33. The method of claim 29 wherein the glycemic control agent or therapy is selected from the compounds of Formula I or Formula II.

34. The method of claim 29 wherein the disorder characterized by impaired glycemic control is type 2 diabetes mellitus.

35. The method of claim 29 wherein the disorder characterized by impaired glycemic control is type 1 diabetes mellitus.

36. The method of claim 29 wherein the disorder characterized by impaired glycemic control is impaired glucose tolerance.

37. The method of claim 29 wherein the disorder characterized by impaired glycemic control is impaired fasting glucose.

38. The method of claim 29 wherein the disorder characterized by impaired glycemic control is Syndrome X.

39. The method of claim 29 wherein the disorder characterized by impaired glycemic control is gestational diabetes.

40. The method of claim 29 wherein the disorder characterized by impaired glycemic control is impaired glucose metabolism (IGM).

41. The method of claim 29 wherein the disorder characterized by impaired glycemic control is a disorder responsive to DPP4 inhibitors.

42. A method of treating a patient with a disorder characterized by impaired glycemic control comprising,
(a) providing genetic counseling to the patient and patients family,
(b) determining the patients genotype for the TCF1 gene at the polymorphism site 483 A>G,
(c) determining the proper therapy for said patient based on results of the genotype determination.

43. A method for optimizing clinical trial design for glycemic control agents, comprising,
(a) determining, for the two copies of the TCF1 gene present in an individual being considered for inclusion in the clinical trial, the identity of the nucleotide pair at the polymorphic site 483 A>G wherein,
(b) if both the nucleotide pairs are CG or if one is AT and one is CG the individual is included in the clinical trial and if the nucleotide pairs are both AT the individual is not included.

44. A method for identifying individuals, with a disorder characterized by impaired glycemic control, who would benefit from drug A vs. B, comprising,
(a) determining, for the two copies of the TCF1 gene present in the individual, the identity of the nucleotide pair at the polymorphic site 483 A>G, wherein,
(b) both the nucleotide pairs are CG or if one is AT and one is CG the individual would benefit from a glycemic control agent or therapy and if the nucleotide pairs are both AT the individual would benefit from alternate glycemic control therapy.

45. A method for determining which individuals, with a disorder characterized by impaired glycemic control, could be treated with a glycemic control agents with reduced side effects, comprising, determining, for the two copies of the TCF1 gene present in the individual, the identity of the nucleotide pair at the polymorphic site 483 A>G, wherein, if both the nucleotide pairs are CG or if one is AT and one is CG the individual can be treated with lower doses of a glycemic control agent with fewer side effects and if the nucleotide pairs are both AT the individual must be treated with higher doses of a glycemic control agent and therefore greater side effects.

46. A method for determining the responsiveness of an individual with a disorder characterized by impaired glycemic control to treatment with a glycemic control agent or therapy, comprising;
(a) determining, for the two copies of the TCF1 gene present in the individual, the identity of a nucleotide pair at a polymorphic site in the region of the TCF1 gene that is in linkage disequilibrium with the polymorphic site at TCF1 483 A>G, and
(b) assigning the individual to a good responder group if the nucleotide pair at a polymorphic site in the region of the TCF1 gene that is in linkage disequilibrium with the polymorphic site at 483 A>G indicates that, at the TCF1 polymorphic site at 483 A>G, both nucleotide pairs are GO or one pair is AT and one pair is GC and to a low responder group if said nucleotide pair indicates that both pairs are AT at the TCF1 483 A>G site.

47. A kit for the identification of a patient's polymorphism pattern at the TCF1 polymorphic site at 483 A>G, said kit comprising a means for determining a genetic polymorphism pattern at the TCF1 polymorphic site at 483 A>G.

48. A kit according to claim 47, further comprising a DNA sample collecting means.

49. A kit according to claim 47, wherein the means for determining a genetic polymorphism pattern at the TCF1 polymorphic site at 483 A>G comprise at least one TCF1 genotyping oligonucleotide.

50. A kit according to claim 47 wherein the means for determining a genetic polymorphism pattern at the TCF1 polymorphic site at 483 A>G comprise two TCF1 genotyping oligonucleotides.

51. A kit according to claim 47, wherein the means for determining a genetic polymorphism pattern at the TCF1 polymorphic site at 483 A>G comprise at least one TCF1 genotyping primer compositor comprising at least one TCF1 genotyping oligonucleotide.

52. A kit according to claim 51, wherein the TCF1 genotyping primer compositor comprises at least two sets of allele specific primer pairs.

53. A kit according to claim 50, wherein the two TCF1 genotyping oligonucleotides are packaged in separate containers.

54. A method according to claim 1, wherein the determination step (a) further comprises the use a kit according to claim 47.

55. A method according to claim 1, wherein said method is performed ex vivo.


SEQUENCE LISTING (4 sequences)

SEQ ID NO: 1 - DNA, 300 bp, Homo sapiens
  prim_transcript (1)...(300): Primer extension assay of TCF1 gene
ggcccagctgattccctccccttccactccaggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagaaccccttcatggccaccatggctcagctgcagagcccccacggtgagcaccctgtgccccacacagcaggagatgatgatagaggttggctgtcaatggatgcaggggaaaggggtgc

SEQ ID NO: 2 - DNA, 26 bp, Homo sapiens
  primer_bind (1)...(26): Invader
ctgagccatggtggccatgaagggga

SEQ ID NO: 3 - DNA, 26 bp, Homo sapiens
  primer_bind (1)...(26): Probe 1
cgcgccgaggttctgggtcacatggc

SEQ ID NO: 4 - DNA, 30 bp, Homo sapiens
  primer_bind (1)...(30): Probe 2
atgacgtggcagacctctgggtcacatggc